SEQUENCE LISTING (16 sequences)

SEQ ID NO: 1 - DNA, 31 bp, Artificial
  Forward Primer (mArom5′/1F)
agtagagctcaaagacaccaccacaaaaatc

SEQ ID NO: 2 - DNA, 30 bp, Artificial
  Reverse Primer (mArom5′/1170R)
taatggtaccagaaagaaggatgcaagcaa

SEQ ID NO: 3 - DNA, 1170 bp, Oryzias latipes
  Medaka ovarian P450 aromatase promoter (1)..(1170)
aaagacaccaccacaaaaatcaaaatgtgaagacgttaacattgacatttactgaaactaaacaattaaaatgttgacacgtgtgtacatgtttgggtgacagtaagtgttcttgtgtccactgaaccttaactaatgaaaaagttgaaaatgtgttagattaattttccttcaaccttttgctaatgtaaatgcaaacagtgcatcactccacagtcactaaatgaaaacataacctgatcatatagatcaggttatgttttctgtaatactgtccagagaccatcttttttaccaacttcagtctcaatgtcatttatgtatgtatatgtatggtgatcagcctgaacagttacaacacacagaaacctactgtacaacgagaaagcctctgtttgtctacactaagcaaaagtctggttatggtggtttcacggtaaaggtttaggtcagttttaaggcattaagaagtttatgattataattatgttctacattatctatatcttgcctcctgctttttcaggattgcaaggcagcttgtatttgaagagaaatccaatctggaacagaaagaacatgcagtatcttttagatagatctttgaatgggcttttttttttaccaggaaactgcagatctgcctttcatataaattctaaaaagctcaaaataaaaaaatctgtgaaaaatatcttagcatgctaacctccagcaccaagtcgaagcattttcaaagattagagaacttttttccttgagtttttccaaacaacaatgtcaaattttacaagatgaggctgcagttgtagctgatactcccgtcatcccatggccctgctgttaggtgcaggatatccgaccaggcttttttgatgttcaaagaaagaagactgcaacctgttacccaactgtcaaacaaaaccccagatcaacctacccccacccgcctgtgtcctgttcattcaaggccataggcaagaactcaaccctccaaggtcttcagtgtattgtttaccctttccttactatgttgtggcttgtgtattaccctgacctggcttgtgaccagctccgactggtataaagagaaaagcgaggaagcttccagtgtttgcacacatcaggttggagcagctgtgctcattgttgcttgcatccttctttctg

SEQ ID NO: 4 - DNA, 29 bp, Artificial
  Forward primer (zArom1F)
ccatcgatatccgttcttatggcaggtga

SEQ ID NO: 5 - DNA, 29 bp, Artificial
  Reverse primer (zArom1571R)
ccatcgatatgtgatggcgaatgagtgtg

SEQ ID NO: 6 - DNA, 21 bp, Artificial
  Forward primer (mAromU1)
agcagctgtgctcattgttgc

SEQ ID NO: 7 - DNA, 21 bp, Artificial
  Reverse primer (mAromL1)
atacatgaagcactggtggtc

SEQ ID NO: 8 - DNA, 1790 bp, Brachydanio rerio
  1790 bp zebrafish ovarian P450 aromatase mRNA (1)..(1790)
ccatcgatccgttcttatggcaggtgatctgctccagccctgtggaatgaagccggtgcgtctcggcgaggctgtggtggatcttcttatccaaagggctcataacggcactgaaagggctcaggacaatgcgtgtggagctacagctacaatactgctgctgctactctgcctgctgctggccatcagacaccatcgaccacacaaatcacacattccaggtccttctttcttttttggtctgggtcctgttgtctcctactgtcggttcatctggtctgggatcgggactgccagcaactactacaacagcaagtatggagacattgtgcgtgtctggatcaatggtgaggaaactctcatcttgagcaggtcgtcagctgtatatcacgtgttaaggaagtctttgtacacttcacgctttggaagtaaactgggtctgcagtgcatcgggatgcatgagcagggcatcatattcaactcaaatgtggctctctggaagaaagtccgtgcattttatgctaaagctctcacaggtccagggcttcagaggactatggagatctgcaccacctccacaaactctcacctggacgatttgtctcagctgacggatgctcaaggacagctggacattcttaacttactgcgatgcatcgtggtggacgtttccaacagactgtttctaggagtcccgctcaatgagcacgatctgcttcagaagattcataaatactttgacacctggcagactgtattaatcaagcctgatgtctacttcagattggactggctgcacagaaagcacaaaagagatgctcaggagttgcaggatgccatcacagctctgatcgagcagaagaaagttcaactggcacacgcagagaaacttgaccacctcgactttacagcagagctgatatttgctcagagccatggagagctgagcgcagagaacgtcaggcagtgtgtgttggagatggtgatcgcggctccagacactctctccatcagtctgttcttcatgctgctgttattaaaacaaaatccagatgtcgagttaaagatcctgcaggaaatggacagtgttttagctggccagagcctccagcactcgcatctgtccaagctgcagatcctggagagttttatcaacgagtctctacgttttcacccggtcgtggacttcaccatgcggcgggcgctggatgatgatgtcatcgagggctacaacgtgaagaaaggaacaaacatcatactgaatgtgggtcgaatgcacagatccgaattcttctccaaacccaatcagttcagtcttgacaacttccataaaaatgttccgagtcgtttcttccagccgttcggatcgggtcctcggtcgtgtgtggggaagcacattgccatggtgatgatgaagtctattctggtggctctgctgtctcgtttctccgtgtgtcctatgaaggcctgtacagtagaaaacatcccgcaaaccaacaacctgtcgcagcagccggtggaggagccgtccagcctcagcgtgtagcttatcctcagaaacactctctgatccacacacactcattcgccatcacatatttatctactgcttctgcttttggatatttgttgaaatgtgtacttcagtgtgaatgtaacatattgctttgatgtatgatattgtgattgtctgttaatttctgtaactaaatgtaactattataatgtatgattttgtgacttaatttctgtaacctttttttattgttaataaaagtattcaaatttga

SEQ ID NO: 9 - DNA, 20 bp, Artificial
  Forward primer (mAromX1.F)
ctgtgctcattgttgcttgc

SEQ ID NO: 10 - DNA, 26 bp, Artificial
  Reverse Primer (mAromX(Sal).R)
gtcgacataggtacgaatctttttcc

SEQ ID NO: 11 - DNA, 25 bp, Artificial
  Forward Primer (mA-i1(Pst).F)
ctgcagataactgtgctcgttgttg

SEQ ID NO: 12 - DNA, 26 bp, Artificial
  Reverse Primer (mAr-i1(Sal).R)
cacagtcgaccaatgtctccatattt

SEQ ID NO: 13 - DNA, 1193 bp, Oryzias latipes
  Medaka ovarian P450 aromatase dsRNA sequence (1)..(1193)
gaattcgcccttctgtgctcattgttgcttgcatccttctttctgattggatctgatccctgcttgtgatcggacaatgtcatcttcatgtctggtggcagagctagtctcaatagccccaaataccactgtaggactcccatccagcatccccatggcaacaaggtccctcatcctgctcgtctgtctcctgttaatggtctggagtcacagtgagaagaaaaccatccctggaccttctttctgtttgggtttgggcccgcttatgtcctatttgagattcatctggactggtactggcacagccagcaactattataacaacaaatatggagacattgttcgagtgtggatcaacagagaggagaccctgatcctcagcagggcatcagcagtgcatcacgttctcaagaacagaaaatacacctcgcgttttggaagcaaacaaggactcagctgcatcggcatgaacgagaagggaatcatatttaacaacaatgtggctttatggaaaaagattcgtacctatgtcgaccaatgtctccatatttgttgttataatagttgctggctgtgccaataccagtccagatgaatctcaaataggacataagcgggcccaaacccaaacagaaagaaggtccagggatggttttcttctcactgtgactccagaccattaacaggagacagacgagcaggatgagggaccttgttgccatggggatgccggatgggagtcctacagtggtatttggggctattgagactagctctgccaccagacatgaagatgacattgtccgatcacaagcagggatcagatcctatcagaaagaaggatgcaagcaacaacgagcacagttatctgcagcaattggatatcaagcttatcgatgatgatccagacatgataagatacattgatgagtttggacaaaccacaactagaatgcagtgaaaaaaatgctttatttgtgaaatttgtgatgctattgctttatttgtaaccattataagctgcaataaacaagttaacaacaacaattgcattcattttatgtttcaggttcagggggaggtgtgggaggttttttaaagcaagtaaaacctctacaaatgtggtatggctgattatgatcctctagatcagatccactagttctagagcggccgccaccgcggtggagctc

SEQ ID NO: 14 - DNA, 6652 bp, Oryzias latipes
  Medaka aromatase driven GFP-zebrafish antisense fusion construct with vector backbone (1)..(6652)
gagctcaaagacaccaccacaaaaatcaaaatgtgaagacgttaacattgacatttactgaaactaaacaattaaaatgttgacacgtgtgtacatgtttgggtgacagtaagtgttcttgtgtccactgaaccttaactaatgaaaaagttgaaaatgtgttagattaattttccttcaaccttttgctaatgtaaatgcaaacagtgcatcactccacagtcactaaatgaaaacataacctgatcatatagatcaggttatgttttctgtaatactgtccagagaccatcttttttaccaacttcagtctcaatgtcatttatgtatgtatatgtatggtgatcagcctgaacagttacaacacacagaaacctactgtacaacgagaaagcctctgtttgtctacactaagcaaaagtctggttatggtggtttcacggtaaaggtttaggtcagttttaaggcattaagaagtttatgattataattatgttctacattatctatatcttgcctcctgctttttcaggattgcaaggcagcttgtatttgaagagaaatccaatctggaacagaaagaacatgcagtatcttttagatagatctttgaatgggcttttttttttaccaggaaactgcagatctgcctttcatataaattctaaaaagctcaaaataaaaaaatctgtgaaaaatatcttagcatgctaacctccagcaccaagtcgaagcattttcaaagattagagaacttttttccttgagtttttccaaacaacaatgtcaaattttacaagatgaggctgcagttgtagctgatactcccgtcatcccatggccctgctgttaggtgcaggatatccgaccaggcttttttgatgttcaaagaaagaagactgcaacctgttacccaactgtcaaacaaaaccccagatcaacctacccccacccgcctgtgtcctgttcattcaaggccataggcaagaactcaaccctccaaggtcttcagtgtattgtttaccctttccttactatgttgtggcttgtgtattaccctgacctggcttgtgaccagctccgactggtataaagagaaaagcgaggaagcttccagtgtttgcacacatcaggttggagcagctgtgctcattgttgcttgcatccttctttctggtaccggtagaaaaaatgagtaaaggagaagaacttttcactggagttgtcccaattcttgttgaattagatggtgatgttaatgggcacaaattttctgtcagtggagagggtgaaggtgatgcaacatacggaaaacttacccttaaatttatttgcactactggaaaactacctgttccatggccaacacttgtcactactttctcttatggtgttcaatgcttttcaagatacccagatcatatgaaacagcatgactttttcaagagtgccatgcccgaaggttatgtacaggaaagaactatatttttcaaagatgacgggaactacaagacacgtgctgaagtcaagtttgaaggtgatacccttgttaatagaatcgagttaaaaggtattgattttaaagaagatggaaacattcttggacacaaattggaatacaactataactcacacaatgtatacatcatggcagacaaacaaaagaatggaatcaaagttaacttcaaaattagacacaacattgaagatggaagcgttcaactagcagaccattatcaacaaaatactccaattggcgatggccctgtccttttaccagacaaccattacctgtccacacaatctgccctttcgaaagatcccaacgaaaagagagaccacatggtccttcttgagtttgtaacagctgctgggattacacatggcatggatgaactatacaaatagcattcgtagaattcggatctgtgcattcgacccacattcagtatgatgtttgttcctttcttcacgttgtagccctcgatgacatcatcatccagcgcccgccgcatggtgaagtccacgaccgggtgaaaacgtagagactcgttgataaaactctccaggatctgcagcttggacagatgcgagtgctggaggctctggccagctaaaacactgtccatttcctgcaggatctttaactcgacatctggattttgttttaataacagcagcatgaagaacagactgatggagagagtgtctggagccgcgatcaccatctccaacacacactgcctgacgttctctgcgctcagctctccatggctctgagcaaatatcagctctgctgtaaagtcgaggtggtcaagtttctctgcgtgtgccagttgaactttcttctgctcgatcagagctgtgatggcatcctgcaactcctgagcatctcttttgtgctttctgtgcagccagtccaatctgaagtagacatcaggcttgattaatacagtctgccaggtgtcaaagtatttatgaatcttctgaagcagatcgtgctcattgagcgggactcctagaaacagtctgttggaaacgtccaccacgatgcatcgcagtaagttaagaatgtccagctgtccttgagcatccgtcagctgagacaaatcgtccaggtgagagtttgtggaggtggtgcagatctccatagtcctctgaagccctggacctgtgagagctttagcataaaatgcacggactttcttccagagagccacatttgagttgaatatgatgccctgctcatgcatcccgatgcactgcagacccagtttacttccaaagcgtgaagtgtacaaagacttccttaacacgtgatatacagctgacgacctgctcaagatgagagtttcctcaccattgatccagacacgcacaatgtctccatacttgctgttgtagtagttgctggcagtcccgatcccagaccagatgaaccgacagtaggagacaacaggacccagaccaaaaaagaaagaaggacctggaatgtgtgatttgtgtggtcgatggtgtctgatggccagcagcaggcagagtagcagcagcagtattgtagctgtagctccacacgcattgtcctgagccctttcagtgccgttatgagccctttggataagaagatccaccacagcctcgccgagacgcaccggcttcattccacagggctggagcagatcacctgccataagaacggatcgatgatgatccagacatgataagatacattgatgagtttggacaaaccacaactagaatgcagtgaaaaaaatgctttatttgtgaaatttgtgatgctattgctttatttgtaaccattataagctgcaataaacaagttaacaacaacaattgcattcattttatgtttcaggttcagggggaggtgtgggaggttttttaaagcaagtaaaacctctacaaatgtggtatggctgattatgatcctctagatcagatccactagttctagagtcgacctgcaggcatgcaagcttgagtattctatagtgtcacctaaatagcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtctaagaaaccattattatcatgacattaacctataaaaataggcgtatcacgaggccctttcgtctcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggctggcttaactatgcggcatcagagcagattgtactgagagtgcaccatatgcggtgtgaaataccgcacagatgcgtaaggagaaaataccgcatcaggaaattgtaagcgttaatattttgttaaaattcgcgttaaatttttgttaaatcagctcattttttaaccaataggccgaaatcggcaaaatcccttataaatcaaaagaatagaccgagatagggttgagtgttgttccagtttggaacaagagtccactattaaagaacgtggactccaacgtcaaagggcgaaaaaccgtctatcagggcgatggcccactacgtgaaccatcaccctaatcaagttttttggggtcgaggtgccgtaaagcactaaatcggaaccctaaagggagcccccgatttagagcttgacggggaaagccggcgaacgtggcgagaaaggaagggaagaaagcgaaaggagcgggcgctagggcgctggcaagtgtagcggtcacgctgcgcgtaaccaccacacccgccgcgcttaatgcgccgctacagggcgcgtccattcgccattcaggctgcgcaactgttgggaagggcgatcggtgcgggcctcttcgctattacgccagctggcgaaagggggatgtgctgcaaggcgattaagttgggtaacgccagggttttcccagtcacgacgttgtaaaacgacggccagtgaattgtaatacgactcactatagggcgaattcgagctc

SEQ ID NO: 15 - DNA, 6198 bp, Oryzias latipes
  Medaka aromatase driven GFP-zebrafish double stranded RNA fusion construct with vector backbone (1)..(6198)
gagctcaaagacaccaccacaaaaatcaaaatgtgaagacgttaacattgacatttactgaaactaaacaattaaaatgttgacacgtgtgtacatgtttgggtgacagtaagtgttcttgtgtccactgaaccttaactaatgaaaaagttgaaaatgtgttagattaattttccttcaaccttttgctaatgtaaatgcaaacagtgcatcactccacagtcactaaatgaaaacataacctgatcatatagatcaggttatgttttctgtaatactgtccagagaccatcttttttaccaacttcagtctcaatgtcatttatgtatgtatatgtatggtgatcagcctgaacagttacaacacacagaaacctactgtacaacgagaaagcctctgtttgtctacactaagcaaaagtctggttatggtggtttcacggtaaaggtttaggtcagttttaaggcattaagaagtttatgattataattatgttctacattatctatatcttgcctcctgctttttcaggattgcaaggcagcttgtatttgaagagaaatccaatctggaacagaaagaacatgcagtatcttttagatagatctttgaatgggcttttttttttaccaggaaactgcagatctgcctttcatataaattctaaaaagctcaaaataaaaaaatctgtgaaaaatatcttagcatgctaacctccagcaccaagtcgaagcattttcaaagattagagaacttttttccttgagtttttccaaacaacaatgtcaaattttacaagatgaggctgcagttgtagctgatactcccgtcatcccatggccctgctgttaggtgcaggatatccgaccaggcttttttgatgttcaaagaaagaagactgcaacctgttacccaactgtcaaacaaaaccccagatcaacctacccccacccgcctgtgtcctgttcattcaaggccataggcaagaactcaaccctccaaggtcttcagtgtattgtttaccctttccttactatgttgtggcttgtgtattaccctgacctggcttgtgaccagctccgactggtataaagagaaaagcgaggaagcttccagtgtttgcacacatcaggttggagcagctgtgctcattgttgcttgcatccttctttctggtaccggtagaaaaaatgagtaaaggagaagaacttttcactggagttgtcccaattcttgttgaattagatggtgatgttaatgggcacaaattttctgtcagtggagagggtgaaggtgatgcaacatacggaaaacttacccttaaatttatttgcactactggaaaactacctgttccatggccaacacttgtcactactttctcttatggtgttcaatgcttttcaagatacccagatcatatgaaacagcatgactttttcaagagtgccatgcccgaaggttatgtacaggaaagaactatatttttcaaagatgacgggaactacaagacacgtgctgaagtcaagtttgaaggtgatacccttgttaatagaatcgagttaaaaggtattgattttaaagaagatggaaacattcttggacacaaattggaatacaactataactcacacaatgtatacatcatggcagacaaacaaaagaatggaatcaaagttaacttcaaaattagacacaacattgaagatggaagcgttcaactagcagaccattatcaacaaaatactccaattggcgatggccctgtccttttaccagacaaccattacctgtccacacaatctgccctttcgaaagatcccaacgaaaagagagaccacatggtccttcttgagtttgtaacagctgctgggattacacatggcatggatgaactatacaaatagcattcgtagaattccgttcttattggcaggtgatctgctccagccctgtggaatgaagccggtgcgtctcggcgaggctgtggtggatcttcttatccaaagggctcataacggcactgaaagggctcaggacaatgcgtgtggagctacagctacaatactgctgctgctactctgcctgctgctggccatcagacaccatcgaccacacaaatcacacattccaggtccttctttcttttttggtctgggtcctgttgtctcctactgtcggttcatctggtctgggatcgggactgccagcaactactacaacagcaagtatggagacattgtgcgtgtctggatcaatggtgaggaaactctcatcttgagcaggtcgtcagctgtatatcacgtgttaaggaagtctttgtacacttcacgctttggaagtaaactgggtctgcagtgcatcgggatgcatgagcagggcatcatattcaactcaaatgtggctctctggaagaaagtcgacgttgctggcagtcccgatcccagaccagatgaaccgacagtaggagacaacaggacccagaccaaaaaagaaagaaggacctggaatgtgtgatttgtgtggtcgatggtgtctgatggccagcagcaggcagagtagcagcagcagtattgtagctgtagctccacacgcattgtcctgagccctttcagtgccgttatgagccctttggataagaagatccaccacagcctcgccgagacgcaccggcttcattccacagggctggagcagatcacctgccataagacggatatctgcagcaattggatatcaagcttatcgatgatgatccagacatgataagatacattgatgagtttggacaaaccacaactagaatgcagtgaaaaaaatgctttatttgtgaaatttgtgatgctattgctttatttgtaaccattataagctgcaataaacaagttaacaacaacaattgcattcattttatgtttcaggttcagggggaggtgtgggaggttttttaaagcaagtaaaacctctacaaatgtggtatggctgattatgatcctctagatcagatccactagttctagagtcgacctgcaggcatgcaagcttgagtattctatagtgtcacctaaatagcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtctaagaaaccattattatcatgacattaacctataaaaataggcgtatcacgaggccctttcgtctcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggctggcttaactatgcggcatcagagcagattgtactgagagtgcaccatatgcggtgtgaaataccgcacagatgcgtaaggagaaaataccgcatcaggaaattgtaagcgttaatattttgttaaaattcgcgttaaatttttgttaaatcagctcattttttaaccaataggccgaaatcggcaaaatcccttataaatcaaaagaatagaccgagatagggttgagtgttgttccagtttggaacaagagtccactattaaagaacgtggactccaacgtcaaagggcgaaaaaccgtctatcagggcgatggcccactacgtgaaccatcaccctaatcaagttttttggggtcgaggtgccgtaaagcactaaatcggaaccctaaagggagcccccgatttagagcttgacggggaaagccggcgaacgtggcgagaaaggaagggaagaaagcgaaaggagcgggcgctagggcgctggcaagtgtagcggtcacgctgcgcgtaaccaccacacccgccgcgcttaatgcgccgctacagggcgcgtccattcgccattcaggctgcgcaactgttgggaagggcgatcggtgcgggcctcttcgctattacgccagctggcgaaagggggatgtgctgcaaggcgattaagttgggtaacgccagggttttcccagtcacgacgttgtaaaacgacggccagtgaattgtaatacgactcactatagggcgaattcgagctc

SEQ ID NO: 16 - DNA, 8286 bp, Oryzias latipes
  pBiT-ds.zAroma ds-zAromatase (1)..(8286)
actagttctagagcggccgcctgcaggaattcggggccgcggaggctggatcggtcccggtgtcttctatggaggtcaaaacagcgtggatggcgtctccaggcgatctgacggttcactaaacgagctctgcttatataggtcgagtttaccactccctatcagtgatagagaaaagtgaaagtcgagtttaccactccctatcagtgatagagaaaagtgaaagtcgagtttaccactccctatcagtgatagagaaaagtgaaagtcgagtttaccactccctatcagtgatagagaaaagtgaaagtcgagtttaccactccctatcagtgatagagaaaagtgaaagtcgagtttaccactccctatcagtgatagagaaaagtgaaagtcgagtttaccactccctatcagtgatagagaaaagtgaaagtcgagctcggtacccgggtcgagtaggcgtgtacggtgggaggcctatataagcagagctcgtttagtgaaccgtcagatcgcctggagacgccatccacgctgttttgacctccatagaagacaccgggaccgatccagcctccgcggccccgaattcgagctcggtacccggggatcctctagtcagaattccgttcttattggcaggtgatctgctccagccctgtggaatgaagccggtgcgtctcggcgaggctgtggtggatcttcttatccaaagggctcataacggcactgaaagggctcaggacaatgcgtgtggagctacagctacaatactgctgctgctactctgcctgctgctggccatcagacaccatcgaccacacaaatcacacattccaggtccttctttcttttttggtctgggtcctgttgtctcctactgtcggttcatctggtctgggatcgggactgccagcaactactacaacagcaagtatggagacattgtgcgtgtctggatcaatggtgaggaaactctcatcttgagcaggtcgtcagctgtatatcacgtgttaaggaagtctttgtacacttcacgctttggaagtaaactgggtctgcagtgcatcgggatgcatgagcagggcatcatattcaactcaaatgtggctctctggaagaaagtcgacgttgctggcagtcccgatcccagaccagatgaaccgacagtaggagacaacaggacccagaccaaaaaagaaagaaggacctggaatgtgtgatttgtgtggtcgatggtgtctgatggccagcagcaggcagagtagcagcagcagtattgtagctgtagctccacacgcattgtcctgagccctttcagtgccgttatgagccctttggataagaagatccaccacagcctcgccgagacgcaccggcttcattccacagggctggagcagatcacctgccataagacggatatctgcagcaattggatatcaagctctgacgcgtgctagcgcggcctcgacgatatctctagactgagaacttcagggtgagtttggggacccttgattgttctttctttttcgctattgaaaaattcatgttatatggagggggcaaagttttcagggtgttgtttagaatgggaagatgtcccttgtatcaccatggaccctcatgataattttgtttctttcactttctactctgttgacaaccattgtctcctcttattttcttttcattttctgtaacttttttcgttaaactttagcttgcatttgtaacgaatttttaaattcactttcgtttatttgtcagattgtaagtactttctctaatcacttttttttcaaggcaatcagggtaattatattgtacttcagcacagttttagagaacaattgttataattaaatgataaggtagaatatttctgcatataaattctggctggcgtggaaatattcttattggtagaaacaactacatcctggtaatcatcctgcctttctctttatggttacaatgatatacactgtttgagatgaggataaaatactctgagtccaaaccgggcccctctgctaaccatgttcatgccttcttctttttcctacagctcctgggcaacgtgctggttgttgtgctgtctcatcattttggcaaagaattcactcctcaggtgcaggctgcctatcagaaggtggtggctggtgtggccaatgccctggctcacaaataccactgagatctttttccctctgccaaaaattatggggacatcatgaagccccttgagcatctgacttctgggtaataaaggaaatttattttcattgcaatagtgtgtgggaattttttgtgtctctcactcggaaggacatatgggagggcaaatcatttaaaacatcagaatgagtatttggtttagagtttggcaacatatgccatatgctggctgccatgaacaaaggtggctataaagaggtcatcagtatatgaaacagccccctgctgtccattccttattccatagaaaagccttgacttgaggttagattttttttatattttgttttgtgttatttttttctttaacatccctaaaattttccttacatgttttactagccagatttttcctcctctcctgactactcccagtcatagctgtccctcttctcttatgaactcgactgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcaatgctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaacacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgcgacaagctttacaatattatatttgtgcatatacattagactagtcagtattaaagccaaatctggagctaatttaacaaaataacttatgattatagtataaaatttgtacacgcaaatttgtaagttaagcaaatatatatatatatatatatatatatatatatatatatatatatatatatatatatatatatatatatatatatatatatatatatccctcaagatattttttattattgttatttttgttactacagggactagagatgtaaagtcagaattattagcccccttgtatattttcccccccatttctgtttaacggaaagcagatttttttaagcagaccttgaaatggcttttaaaaaattaaaaacttgttattttctagccgaaataaaacaaataagactttctcccttgctctgataaaaatcatttgggaaatattaaaaaaagaacacaatttcaaaggggcactaataattctgacatcaactttaaattttatttatttatcttttggtaactacgacgacaagagatgtaatttagctttatagctatggcacaacatgtcatgttgtagctacattgtcccagaataagtaaataaaagaatattcggctttatacaagtctaaaatagttttacataaaatgttagatcattttaaaacgtttaaagacaacacattgcaataacaaatcaattaaatgaaacctaaaataacgttaacatttacccttcactataaattactatacatgattttaaacagaagatatatccttataaatactgaaaaaatactcaaatacaaatgtagataatttaaattagtgcgcatttaaatttaggatttgtttaaccatacttcagtctcaattgtattgcgtatacattacattctcgttcaaattactaacatgtttacataggataatacataaaatatgccccatgcaggggaaattcggtccatccgcgcgcgcagagtgtgggcatgttcaaacgcttgaatggagagagcgcggcatcattgtgacatcatcagacaacaaaaagccttgcgctcgcgcagcgaagcgctccaatcaatggcacagacgcggcgcgtgctgcacgcagagatgagtctccaaacagccacggaaaacttctgctgaccacaagtttttgatttctttaaaacaaaaacaaaaaatgacaaatccaggattgtgcgatctcgcgctgtcacttttgggattgctgctgtctttgacctgagcgctcgcgcacttcattagagtttagtagagtctagtctgaagtgttgcacaagtatgaacaagaagaggcgacttgagctgcgacgactctctgtcgtgggataaaaaaatcgcttgtggattaaaacacgaattcatatgtctagattagataaaagtaaagtgattaacagcgcattagagctgcttaatgaggtcggaatcgaaggtttaacaacccgtaaactcgcccagaagctaggtgtagagcagcctacattgtattggcatgtaaaaaataagcgggctttgctcgacgccttagccattgagatgttagataggcaccatactcacttttgccctttagaaggggaaagctggcaagattttttacgtaataacgctaaaagttttagatgtgctttactaagtcatcgcgatggagcaaaagtacatttaggtacacggcctacagaaaaacagtatgaaactctcgaaaatcaattagcctttttatgccaacaaggtttttcactagagaatgcattatatgcactcagcgctgtggggcattttactttaggttgcgtattggaagatcaagagcatcaagtcgctaaagaagaaagggaaacacctactactgatagtatgccgccattattacgacaagctatcgaattatttgatcaccaaggtgcagagccagccttcttattcggccttgaattgatcatatgcggattagaaaaacaacttaaatgtgaaagtgggtccgcgtacagccgcgcgcgtacgaaaaacaattacgggtctaccatcgagggcctgctcgatctcccggacgacgacgcccccgaagaggcggggctggcggctccgcgcctgtcctttctccccgcgggacacacgcgcagactgtcgacggcccccccgaccgatgtcagcctgggggacgagctccacttagacggcgaggacgtggcgatggcgcatgccgacgcgctagacgatttcgatctggacatgttgggggacggggattccccgggtccgggatttaccccccacgactccgccccctacggcgctctggatatggccgacttcgagtttgagcagatgtttaccgatgcccttggaattgacgagtacggtgggtagggggcgcgaggatccagacatgataagatacattgatgagtttggacaaaccacaactagaatgcagtgaaaaaaatgctttatttgtgaaatttgtgatgctattgctttatttgtaaccattataagctgcaataaacaagttaacaacaacaattgcattcattttatgtttcaggttcagggggaggtgtgggaggttttttaaagcaagtaaaacctctacaaatgtggtatggctgattatgatcctgcaagcctcgtcgtctggccggaccacgctatctgtgcaaggtccccggacgcgcgctccatgagcagagcgcccgccgccgaggcaagactcgggcggcgccctgcccgtcccaccaggtcaacaggcggtaaccggcctcttcatcgggaatgcgcgcgaccttcagcatcgccggcatgtcccctggcggacgggaagtatcagctcgaccaagcttgatatcgaattcttacttgtacagctcgtccatgccgagagtgatcccggcggcggtcacgaactccagcaggaccatgtgatcgcgcttctcgttggggtctttgctcagggcggactgggtgctcaggtagtggttgtcgggcagcagcacggggccgtcgccgatgggggtgttctgctggtagtggtcggcgagctgcacgctgccgtcctcgatgttgtggcggatcttgaagttcaccttgatgccgttcttctgcttgtcggccatgatatagacgttgtggctgttgtagttgtactccagcttgtgccccaggatgttgccgtcctccttgaagtcgatgcccttcagctcgatgcggttcaccagggtgtcgccctcgaacttcacctcggcgcgggtcttgtagttgccgtcgtccttgaagaagatggtgcgctcctggacgtagccttcgggcatggcggacttgaagaagtcgtgctgcttcatgtggtcggggtagcggctgaagcactgcacgccgtaggtcagggtggtcacgagggtgggccagggcacgggcagcttgccggtggtgcagatgaacttcagggtcagcttgccgtaggtggcatcgccctcgccctcgccggacacgctgaacttgtggccgtttacgtcgccgtccagctcgaccaggatgggcaccaccccggtgaacagctcctcgcccttgctcaccatccgcggggatcc

(19) United States
(12) Patent Application Publication
(10) Pub. No.: US 2004/0073959 A1
(43) Pub. Date: Apr. 15, 2004

(54) Genetic control of sex ratio in animal populations

(76) Inventors: Ronald Thresher, Ridgeway (AU); Peter Grewe, West Hobart (AU); Jawahar Patil, Hobart (AU); Lyn Hinds, Kaleen (AU)

Correspondence Address: FOLEY AND LARDNER, SUITE 500, 3000 K STREET NW, WASHINGTON, DC, 20007, US

(21) Appl. No.: 10/398,482

(22) PCT Filed: Oct. 9, 2001
(86) PCT No.: PCT/AU01/01264

(30) Foreign Application Priority Data
Oct. 9, 2000 (AU) PR 0646

(51) Int. Cl. A01K 67/00

(52) U.S. Cl. 800/8

(57) ABSTRACT

The invention provides a nucleic acid construct, which may be inserted into the genome of a target organism. The construct consists of a promoter that is activated during the sex-determining stage of embryonic development/or gametogenesis, and a blocker that inhibits expression of a gene critical for sex differentiation.

### FIELD OF INVENTION

This application is concerned with the control of sex ratios in animal populations, and its use in preventing the spread and reducing the impacts of exotic animals. In particular, the present invention relates to constructs that can be bred into a pest population that distort its operational sex ratio, and lead to reproductive failure, population decline and potentially eradication. The genetic technique also has applications in animal husbandry, in producing single sex lines.

### BACKGROUND OF THE INVENTION

Exotic pests are one of the world's major environmental problems. Goats, cats, rabbits and carp are only the more prominent of hundreds of species traded internationally for recreation or agriculture that have escaped into the wild and formed destructive populations. Exotic species introduced accidentally in ballast water, international freight and through other vectors continue to invade and distort ecosystems world-wide. Terrestrial, freshwater and marine ecosystems are all conspicuously degraded by these species, to the extent that public concern over exotic pest animals has become a major issue world-wide.

There is a long history of attempts to manage pest populations. Three broad approaches have been developed, which have mixed success rates. These approaches are 1) physical removal, which includes poisoning and other biocides, 2) environmental remediation, which is based on the hypothesis that exotic species are only a problem in disturbed environments, and 3) biological control, which usually involves the introduction of a pathogen or predator to attack and thereby control the target pest population. None of these approaches are universally applicable, and all three have very severe limitations. A good example is the cane toad, Bufo marinus, which has been broadly introduced as a control agent against pest insect populations, but which has become a problem species in its own right. The cane toad is largely inedible, because of toxin glands in its skin, and hence resists impacts of predation; despite extensive studies, no effective pathogen, disease agent or biocide has been found that can be used against it without a high risk of damaging desirable native amphibian species; and the history of the toad shows that it is largely unaffected by simple environmental manipulations. At this stage, no effective control strategy is in place for the cane toad, for exotic fish species like carp and tilapia, and for a myriad other high profile pests for which historically useful approaches have proven unsuccessful or impractical. Many of these species are currently completely unmanaged and out of control.

Similar problems exist in managing some native animal populations that nonetheless constitute significant economic, ecological and health risks. One of the most prominent examples are the snails in the genera Biomphalaria and Bulinus, which are intermediate hosts for the parasitic worms that cause the schistosomiasis. Numerous attempts have been made to control rates of human infection in Africa, South America and Asia by managing, if not preferably eradicating the host snails, in attempts to break the life cycle of the parasite (Lardans and Drissous, 1998). Other snail species act as intermediate hosts for other animal parasites and some other human diseases, such as Eosinophilous meningoencephalitis in Hawaii, which is caused by the nematode Angiostrogylus cantonensis and vectored by several species of introduced snails. At this stage, there are no effective strategies for managing these diseases through management of the intermediate host populations.

Accordingly, there is still a need to provide methods for controlling the impacts of, if not actually eradicating a number of highly destructive exotic and native pest species.

We have developed such a method that can be applied to amphibians, fishes and molluscs and possibly other animals. We have designed certain genetic constructs that once introduced into a target population progressively skew its sex ratio, ultimately to the point where the population's reproductive output begins to decline. This in turn leads to reduced population size, and can even lead to local extinction (Hamilton, 1967; Werren, et al., 1981).

Accordingly, some of the major benefits that this “daughterless” construct would offer over existing pest management methods include:

1. Provision of a species-specific, and hence inherently safe, method for managing exotic pest populations. The genetic approach avoids the need for environmentally destructive poisons or the introduction of pathogens, diseases or predators that may have severe damaging affects on non-target species. The long-term progressive decline in pest populations provided by use of the “daughterless” construct eliminates collateral damage due to, for example, the need to dispose of large masses of dead animals following release of a biocide.

2. Provision of a control method for exotic pest species, like the cane toad, carp and Giant African Snail, for which no effective options are currently available.

3. Provision of a control method for native and exotic species that act as vectors for human and animal diseases, such as the snails in the genus Biomphalaria which are intermediate hosts of schistosomes and for which no effective control options are available.

4. Provision of a humane, long-term method of pest control, that does not involve undue suffering on the part of the managed pest.

5. Provision of a very cost-effective means of pest control. Long-term control and, potentially, eradication can be achieved by low cost and low effort programs of stocking out animals carrying the genetic construct, which integrate and spread in the pest population.

The genetic technique we have developed results in self-propagating all male lines, through introduction into the target population of a gene construct that causes bi-potential embryonic gonads to develop only as fully functional males. Accordingly, this technique could also be used to produce all male offspring in the husbandry of reptiles, amphibians, fish, molluscs and some other invertebrates. Male offspring do not invest large amounts of energy in gonad development (ripe male gonads are much smaller than ripe female gonads) and hence are often preferred in animal husbandry, such as fish mariculture. Application of this invention allows the farming of all-male animals, for which alternative techniques to produce single sex broods are not available or are not reliable, with a consequent increase in productivity and a reduced likelihood of establishment of feral populations by escaped animals.

### SUMMARY OF THE INVENTION

In its most general aspect, the invention disclosed herein provides a nucleic acid construct which may be inserted into the genome of any target organism. The construct consists of a promoter, that is activated during the sex-determining stage of embryonic development and/or gametogenesis, and a blocker, that inhibits expression of a gene critical for sex differentiation.

Accordingly, in a first aspect, the present invention provides a construct for modifying phenotypic sex in animals, comprising:

a) a first nucleic acid molecule, which is transiently activated in a defined spatio-temporal pattern, and which is operably linked to

b) a second nucleic acid molecule, which encodes a blocker molecule that alters normal sexual development in the animal.

Each of the first and second nucleic acids may be genomic DNA, cDNA, RNA, or a hybrid molecule thereof. It will be clearly understood that the term nucleic acid molecule encompasses a full-length molecule, or a biologically active fragment thereof.

Preferably the first nucleic acid molecule is a DNA molecule encoding a promoter region. More preferably the promoter is activated only during embryonic development and/or gametogenesis, and is expressed in a spatio-temporal domain coincident with sex determination. Most preferably this DNA molecule has the nucleotide sequence shown in SEQ ID NO:3.

Preferably the second nucleic acid molecule encodes a blocker molecule selected from the group consisting of antisense RNA, double-stranded RNA (dsRNA), sense RNA or ribozyme. More preferably the molecule is antisense RNA or dsRNA. Most preferably this DNA molecule has the nucleotide sequence shown in SEQ ID NO:8 and 13.

Samples of the constructs, incorporating both the first nucleic acid molecule (the promoter) and the second nucleic acid molecule (the blocker), as shown in SEQ. ID No. 14 and SEQ. ID. NO. 19, were deposited at the Australian Government Analytical Laboratories on Oct. 4, 2000, and accorded the accession numbers NM00/14911 and NM00/14907, respectively.

In a second aspect, the present invention provides a nucleic acid molecule, which encodes a promoter and is transiently activated in a defined spatio-temporal pattern. More preferably, the promoter is active only during a narrow window during embryogenesis or larval development. Most preferably the nucleic acid is a promoter having a nucleotide sequence as shown in SEQ ID NO:3.

In a third aspect, the present invention provides a nucleic acid molecule, which encodes a promoter having:

a) a nucleotide sequence as shown in SEQ ID NO:3; or

b) a biologically active fragment of the sequence in a); or

c) a nucleic acid molecule which has at least 75% sequence homology to the sequence in a) or b); or

d) a nucleic acid molecule which is capable of hybridizing to the sequence in a) or b) under stringent conditions.

In a fourth aspect, the present invention provides a nucleic acid molecule that encodes the coding region of a gene including:

a) a nucleotide sequence as shown in SEQ ID NO:8 or SEQ. ID NO. 13; or

b) a biologically active fragment of any one of the sequences in a); or

c) a nucleic acid molecule which has at least 75% sequence homology with the sequences disclosed in a) or b); or

d) a nucleic acid molecule that is capable of binding to any one of the sequences disclosed in a) or b) under stringent conditions.

In a fifth aspect, the present invention provides a nucleic acid molecule which encodes a blocker molecule wherein the blocker molecule is capable of altering normal sexual development in an animal, leading to sterility or an alteration of phenotypic sex.

Preferably the blocker molecule is selected from the group consisting of antisense RNA, dsRNA, sense RNA and ribozyme. More preferably the molecule is dsRNA. Most preferably the blocker molecule is encoded, or partially encoded, by SEQ ID NO:8 and 13.

In an sixth aspect, the present invention provides a construct for altering-sexual development in animals, comprising:

a) a first nucleic acid molecule, which is transiently activated in a defined spatio-temporal pattern, and which is operably linked to

b) a second nucleic acid molecule, which encodes a blocker molecule wherein activation of said first nucleic acid molecule controls the expression of the second nucleic acid which induces sterility or modifies phenotypic sex in the animal.

In a seventh aspect, the present invention provides a method of altering phenotypic sex in animals comprising the steps of:

1) stably transforming an animal cell with a construct according to the invention; and

2) implanting the cell into a host organism, whereby a whole animal develops from the implanted cell.

Preferably, the stable transformation is effected by microinjection, transfection or infection, wherein the construct stably integrates into the genome by homologous recombination.

In an eighth aspect, the present invention provides a transgenic animal stably transformed with a construct according to the invention.

Preferably the host organism is of the same genus as the transformed cell. More preferably the host organism is any animal, including vertebrates and invertebrates. Most preferably the host organism is selected from the group consisting of fish, amphibians and molluscs. Fish include; but are not limited to, zebrafish, European carp, salmon, mosquito fish, tench, lampreys, round gobies, tilapia and trout. Amphibians include; but are not limited to, cane toads and bull frogs. Molluscs include; but are not limited to, Pacific oysters, zebra mussels, striped mussels, New Zealand screw shells, the Golden Apple Snail, the Giant African Snail, and the disease vectoring snails in the genera Biomphalaria and Bulinus.

Modified and variant forms of the constructs may be produced in vitro, by means of chemical or enzymatic treatment, or in vivo by means of recombinant DNA technology. Such constructs may differ from those disclosed, for example, by virtue of one or more nucleotide substitutions, deletions or insertions, but substantially retain a biological activity of the construct or nucleic acid molecule of this invention.

### BRIEF DESCRIPTION OF THE FIGURES

FIG. 1 shows the frequency distribution of male offspring carrying different numbers of Sex Differentiating Constructs (SDC) (N) for ten generations, from a founder male carrying 32 copies of the SDC, homozygous on 16 chromosome pairs.

FIG. 2 shows the effect of different copy number (N) of Sex Differentiating Constructs (SDC) in a stocked animal on the proportion of males in its offspring for ten generations.

FIG. 3 shows the parameter values, distribution of age-specific harvesting, natural mortality and sexual maturity, and Ricker curve for the basic carp population model.

FIG. 4 shows the effect of annual stockings of different numbers of juvenile carp containing copies of SDCs that result in all male broods through five generations.

FIG. 5 shows the effects of stocking juvenile carp carrying different number of chromosomes carrying SDCs.

FIG. 6 shows differential effects of stocking juvenile carp carrying sex differentiating constructs versus those carrying constructs that render females infertile.

FIG. 7 shows the effect of harvesting adults on rate of population decline for conditions of annual stocking of 50 juveniles carrying SDCs to produce all-male broods through 5 generations. in recruitment on the efficacy of a 2.5% stocking regime on controlling carp population dynamics

FIG. 9 shows a plasmid map of pmAr5′-GFP. The transcriptional unit consists of the modified GFP coding sequences (Cormac et al., 1996), under the regulation of a 1170 bp medaka ovarian P450 aromatase promoter.

FIG. 10 shows the medaka ovarian P450 aromatase promoter-driven GFP expression in zebrafish embryo at about 31 hpi.

FIG. 11 shows a plasmid map of the GFP-zebrafish antisense fusion construct (pmA5′GzAn), driven by medaka ovarian P450 aromatase promoter.

FIG. 12 shows a plasmid map of GFP-double stranded zebrafish RNA fusion construct, driven by medaka ovarian P450 aromatase promoter.

FIG. 13 shows a plasmid map of a single construct containing Tet-off and the tet-responsive element for regulation of eGFP and aromatase blocker gene sequence.

### DETAILED DESCRIPTION OF THE INVENTION

The practice of the present invention employs, unless otherwise indicated, conventional molecular biology, microbiology, and recombinant DNA techniques within the skill of the art. Such techniques are well known to the skilled worker, and are explained fully in the literature. See, eg., “DNA Cloning: A Practical Approach,” Volumes I and II (D. N. Glover, ed., 1985); “Oligonucleotide Synthesis” (M. J. Gait, ed., 1984); “Nucleic Acid Hybridization” (B. D. Hames & S. J. Higgins, eds., 1985); “Transcription and Translation” (B. D. Hames & S. J. Higgins, eds., 1984); “Animal Cell Culture” (R. I. Freshney, ed., 1986); “Immobilized Cells and Enzymes” (IRL Press, 1986); B. Perbal, “A Practical Guide to Molecular Cloning” (1984), and Sambrook, et al., “Molecular Cloning: a Laboratory Manual” 12^{th} edition (1989).

Definitions

The description that follows makes use of a number of terms used in recombinant DNA technology. In order to provide a clear and consistent understanding of the specification and claims, including the scope given such terms, the following definitions are provided.

A “nucleic acid molecule” or “polynucleic acid molecule” refers herein to deoxyribonucleic acid and ribonucleic acid in all their forms, i.e., single and double-stranded DNA, cDNA, mRNA, and the like.

A “double-stranded DNA molecule” refers to the polymeric form of deoxyribonucleotides (adenine, guanine, thymine, or cytosine) in its normal, double-stranded helix. This term refers only to the primary and secondary structure of the molecule, and does not limit it to any particular tertiary forms. Thus this term includes double-stranded DNA found, inter alia, in linear DNA molecules (e.g., restriction fragments), viruses, plasmids, and chromosomes. In discussing the structure of particular double-stranded DNA molecules, sequences may be described herein according to the normal convention of giving only the sequence in the 5′ to 3′ direction along the non-transcribed strand of DNA (i.e., the strand having a sequence homologous to the mRNA).

A DNA sequence “corresponds” to an amino acid sequence if translation of the DNA sequence in accordance with the genetic code yields the amino acid sequence (i.e., the DNA sequence “encodes” the amino acid sequence).

One DNA sequence “corresponds” to another DNA sequence if the two sequences encode the same amino acid sequence.

Two DNA sequences are “substantially similar” when at least about 85%, preferably at least about 90%, and most preferably at least about 95%, of the nucleotides match over the defined length of the DNA sequences. Sequences that are substantially similar can be identified in a Southern hybridization experiment, for example under stringent conditions as defined for that particular system. Defining appropriate hybridization conditions is within the skill of the art. See e.g., Sambrook et al., “Molecular Cloning: a Laboratory Manual” 12^{th} edition (1989), vols. I, II and III. Nucleic Acid Hybridization. However, ordinarily, “stringent conditions” for hybridization or annealing of nucleic acid molecules are those that

(1) employ low ionic strength and high temperature for washing, for example, 0.015 M NaCl/0.0015 M sodium citrate/0.1% sodium dodecyl sulfate (SDS) at 50° C., or

(2) employ during hybridization a denaturing agent such as formamide, for example, 50% (vol/vol) formamide with 0.1% bovine serum albumin/0.1% Ficoll/0.1% polyvinylpyrrolidone/50 mM sodium phosphate buffer at pH 6.5 with 750 mM NaCl, 75 mM sodium citrate at 42° C.

Another example is use of 50% formamide, 5×SSC (0.75 M NaCl, 0.075 M sodium citrate), 50 mM sodium phosphate (pH 6.8), 0.1% sodium pyrophosphate, 5×Denhardt's solution, sonicated salmon sperm DNA (50 □g/mL), 0.1% SDS, and 10% dextran sulfate at 42° C., with washes at 42° C. in 0.2×SSC and 0.1% SDS.

A “heterologous” region or domain of a DNA construct is an identifiable segment of DNA within a larger DNA molecule that is not found in association with the larger molecule in nature. Thus, when the heterologous region encodes a mammalian gene, the gene will usually be flanked by DNA that does not flank the mammalian genomic DNA in the genome of the source organism. Another example of a heterologous region is a construct where the coding sequence itself is not found in nature (e.g., a cDNA where the genomic coding sequence contains introns, or synthetic sequences having codons different than the native gene). Allelic variations or naturally occurring mutational events do not give rise to a heterologous region of DNA as defined herein.

A “gene” includes all the DNA sequences associated with the promoter and coding region and non-coding region such as introns and 5′ and 3′ non-coding sequences and enhancer elements.

A “coding region” is an in-frame sequence of codons from the start codon, normally ATG, to the stop codon TAA, and which may or may not include introns. A “coding sequence” is an in-frame sequence of codons that correspond to or encode a protein or peptide sequence. Two coding sequences correspond to each other if the sequences or their complementary sequences encode the same amino acid sequences. A coding sequence in association with appropriate regulatory sequences may be transcribed and translated into a polypeptide in vivo. A polyadenylation signal and transcription termination sequence will usually be located 3′ to the coding sequence.

A “promoter sequence” is a DNA regulatory region capable of binding RNA polymerase in a cell and initiating transcription of a downstream (3′direction) coding sequence. A coding sequence is “under the control” of the promoter sequence in a cell when RNA polymerase which binds the promoter sequence transcribes the coding sequence into MRNA, which is then in turn translated into the protein encoded by the coding sequence.

For the purposes of the present invention, the promoter sequence is bounded at its 3′ terminus by the translation start codon of a coding sequence, and extends upstream to include the minimum number of bases or elements necessary to initiate transcription at levels detectable above background. Within the promoter sequence will be found a transcription initiation site (conveniently defined by mapping with nuclease S1), as well as protein binding domains (consensus sequences) responsible for the binding of RNA polymerase. Eukaryotic promoters will often, but not always, contain “TATA” boxes and “CAT” boxes, prokaryotic promoters contain Shine-Delgarno sequences in addition to the −10 and −35 consensus sequences.

A cell has been “transformed” by exogenous DNA when such exogenous DNA has been introduced inside the cell wall. Exogenous DNA may or may not be integrated (covalently linked) to chromosomal DNA making up the genome of the cell. In prokaryotes and yeast, for example, the exogenous DNA may be maintained on an episomal element such as a plasmid. With respect to eukaryotic cells, a stably transformed cell is one in which the exogenous DNA is inherited by daughter cells through chromosome replication. This stability is demonstrated by the ability of the eukaryotic cell to establish cell lines or clones comprised of a population of daughter cells containing the exogenous DNA.

“Integration” of the DNA may be effected using non-homologous recombination following mass transfer of DNA into the cells using microinjection, biolistics, electroporation or lipofection. Alternative methods such as homologous recombination, and or restriction enzyme mediated integration (REMI) or transposons are also encompassed, and may be considered to be improved integration methods.

A “clone” is a population of cells derived from a single cell or common ancestor by mitosis.

“Cell, ” “host cell,” “cell line,” and “cell culture” are used interchangeably herewith and all such terms should be understood to include progeny. A “cell line” is a clone of a primary cell that is capable of stable growth in vitro for many generations. Thus the words “transformants” and “transformed cells” include the primary subject cell and cultures derived therefrom, without regard for the number of times the cultures have been passaged. It should also be understood that all progeny might not be precisely identical in DNA content, due to deliberate or inadvertent mutations.

Vectors are used to introduce a foreign substance, such as DNA, RNA or protein, into an organism. Typical vectors include recombinant viruses (for DNA) and liposomes (for protein). A “DNA cloning vector” is an autonomously replicating DNA molecule, such as plasmid, phage or cosmid. Typically the DNA cloning vector comprises one or a small number of restriction endonuclease recognition sites, at which such DNA sequences may be cut in a determinable fashion without loss of an essential biological function of the vector, and into which a DNA fragment may be spliced in order to bring about its replication and cloning. The cloning vector may also comprise a marker suitable for use in the identification of cells transformed with the cloning vector.

An “expression vector” is similar to a DNA cloning vector, but contains regulatory sequences which are able to direct protein synthesis by an appropriate host cell. This usually means a promoter to bind RNA polymerase and initiate transcription of mRNA, as well as ribosome binding sites and initiation signals to direct translation of the MRNA into a polypeptide. Incorporation of a DNA sequence into an expression vector at the proper site and in correct reading frame, followed by transformation of an appropriate host cell by the vector, enables the production of mRNA corresponding to the DNA sequence, and usually of a protein encoded by the DNA sequence.

“Plasmids” are DNA molecules that are capable of replicating within a host cell, either extrachromosomally or as part of the host cell chromosome(s), and are designated by a lower case “p” preceded and/or followed by capital letters and/or numbers. The starting plasmids herein are commercially available, are publicly available on an unrestricted basis, or can be constructed from such available plasmids by methods disclosed herein and/or in accordance with published procedures. In certain instances, as will be apparent to the ordinarily skilled worker, other plasmids known in the art may be used interchangeably with plasmids described herein.

“Control sequences” refers to DNA sequences necessary for the expression of an operably linked nucleotide coding sequence in a particular host cell. The control sequences suitable for expression in prokaryotes, for example, include origins of replication, promoters, ribosome binding sites, and transcription termination sites. The control sequences that are suitable for expression in eukaryotes, for example, include origins of replication, promoters, ribosome binding sites, polyadenylation signals, and enhancers.

An “exogenous” element is one that is foreign to the host cell, or is homologous to the host cell but in a position within the host cell in which the element is ordinarily not found.

“Digestion” of DNA refers to the catalytic cleavage of DNA with an enzyme that acts only at certain locations in the DNA. Such enzymes are called restriction enzymes or restriction endonucleases, and the sites within DNA where such enzymes cleave are called restriction sites. If there are multiple restriction sites within the DNA, digestion will produce two or more linearized DNA fragments (restriction fragments). The various restriction enzymes used herein are commercially available,and their reaction conditions, cofactors, and other requirements as established by the enzyme manufacturers are used. Restriction enzymes are commonly designated by abbreviations composed of a capital letter followed by other letters representing the microorganism from which each restriction enzyme originally was obtained and then a number designating the particular enzyme. In general, about 1 μg of DNA is digested with about 1-2 units of enzyme in about 20 μl of buffer solution. Appropriate buffers and substrate amounts for particular restriction enzymes are specified by the manufacturer, and/or are well known in the art.

“Recovery” or “isolation” of a given fragment of DNA from a restriction digest typically is accomplished by separating the digestion products, which are referred to as “restriction fragments,” on a polyacrylamide or agarose gel by electrophoresis, identifying the fragment of interest on the basis of its mobility relative to that of marker DNA fragments of known molecular weight, excising the portion of the gel that contains the desired fragment, and separating the DNA from the gel, for example by electroelution.

“Ligation” refers to the process of forming phosphodiester bonds between two double-stranded DNA fragments. Unless otherwise specified, ligation is accomplished using known buffers and conditions with 10 units of T4 DNA ligase per 0.5 μg of approximately equimolar amounts of the DNA fragments to be ligated.

“Oligonucleotides” are short-length, single- or double-stranded polydeoxynucleotides that are chemically synthesized by known methods (involving, for example, triester, phosphoramidite, or phosphonate chemistry), such as described by Engels et al., Agnew. Chem. Int. Ed. Engl. 28:716-734 (1989). They are then purified, for example, by polyacrylamide gel electrophoresis.

“Polymerase chain reaction,” or “PCR,” as used herein generally refers to a method for amplification of a desired nucleotide sequence in vitro, as described in U.S. Pat. No. 4,683,195. In general, the PCR method involves repeated cycles of primer extension synthesis, using two oligonucleotide primers capable of hybridizing preferentially to a template nucleic acid. Typically, the primers used in the PCR method will be complementary to nucleotide-sequences within the template at both ends of or flanking the nucleotide sequence to be amplified, although primers complementary to the nucleotide sequence to be amplified also may be used. See Wang et al., in PCR Protocols, pp.70-75 (Academic Press, 1990); Ochman et al., in PCR Protocols, pp. 219-227; Triglia, et al., Nuc. Acids Res. 16:8186 (1988). “PCR cloning” refers to the use of the PCR method to amplify a specific desired nucleotide sequence that is present amongst the nucleic acids from a suitable cell or tissue source, including total genomic DNA and cDNA transcribed from total cellular RNA. See Frohman et al., Proc. Nat. Acad. Sci. USA 85:8998-9002 (1988); Saiki et al., Science 239:487-492 (1988); Mullis et al., Meth. Enzymol. 155:335-350 (1987).

“mAR promoter” refers to a promoter encoded by the nucleotide sequence set forth in SEQ ID NO.:3. “Blocker molecule” refers to either antisense RNA, dsRNA, sense RNA or DNA that preferably encodes the aromatase expression gene and includes the sequences shown in SEQ ID NO:8 and SEQ ID NO:13. However, it will be appreciated by those skilled in the art that any nucleic acid molecule capable of preventing normal sexual development, leading to sterility or alteration of expressed phenotypic sex, is encompassed. Accordingly, the terms “blocker molecule RNA” and “blocker molecule DNA” as used herein are interchangeable depending upon whether it is a species of RNA or DNA, that is being addressed. Sequence variants of mAR promoter and blocker molecule may be made synthetically, for example, by site-directed or PCR mutagenesis, or may exist naturally, as in the case of allelic forms and other naturally occurring variants of the nucleotide sequences set forth in SEQ ID NO.:3 or SEQ ID NO:8 and SEQ ID NO:13, respectively, that may occur in fish and other animal species.

Aromatase blocker molecule nucleotide sequence variants are included within the scope of the invention, provided that they are functionally active. “Functionally active” and “functional activity” means that the blocker molecule variants are capable of blocking normal sexual development or altering the expressed phenotypic sex in an animal. Therefore, aromatase blocker molecule nucleotide sequence variants generally will share at least about 75% with the nucleotide sequences set forth in SEQ ID NO.:8 or SEQ ID NO.:13, after aligning the sequences to provide for maximum homology, as determined, for example, by the Fitch et al., Proc. Nat. Acad. Sci. USA 80:1382-1386 (1983), version of the algorithm described by Needleman et al., J. Mol. Biol. 48:443-453 (1970).

Nucleotide sequence variants of the aromatase blocker molecule are prepared by introducing appropriate nucleotide changes into blocker molecule DNA, or by in vitro synthesis. Such variants include deletions from, or insertions or substitutions of, nucleotides within the blocker molecule nucleotide sequences set forth in SEQ ID NO:8 and SEQ ID NO:13. Any combination of deletion, insertion, and substitution may be made to arrive at a nucleotide sequence variant of blocker molecule provided that such variants possess the desired characteristics described herein. Changes that are made in the nucleotide sequences set forth in SEQ ID NO:8 and SEQ ID NO:13, respectively, to arrive at nucleotide sequence variants of blocker molecule also may result in further modifications of the blocker molecule upon their activation in host cells.

There are two principal variables in the construction of nucleotide sequence variants of the aromatase blocker molecule nucleic acid: the location of the mutation site and the nature of the mutation. These are variants from the nucleotide sequences set forth in SEQ ID NO:8 and SEQ ID NO:13 and may represent naturally occurring allelic forms of molecule or predetermined mutant forms of blocker molecule made by mutating blocker molecule DNA, either to arrive at an allele or a variant not found in nature. In general, the location and nature of the mutation chosen will depend upon the blocker molecule characteristic to be modified.

Nucleotide sequence deletions generally range from about 1 to 30 nucleotides, more preferably about 1 to 10 nucleotides, and are typically contiguous.

Nucleotide sequence insertions include fusions ranging in length from one nucleotide to hundreds of nucleotides, as well as intrasequence insertions of single or multiple nucleotides. Intrasequence insertions (i.e., insertions made within the nucleotide sequences set forth in SEQ ID NO:8 and SEQ ID NO:13) may range generally from about 1 to 10 nucleotides, more preferably 1 to 5, most preferably 1 to 3.

The third group of variants are those in which nucleotides in the nucleotide sequences set forth in SEQ ID NO:8 and SEQ ID NO:13 have been substituted with other nucleotides. Preferably one to four, more preferably one to three, even more preferably one to two, and most preferably only one nucleotide has been removed and a different nucleotide inserted in its place. The sites of greatest interest for making such substitutions are those sites that are likely to be important to the functional activity of the blocker molecule.

Blocker molecule DNA is obtained by in vitro synthesis. Identification of blocker molecule DNA within a cDNA or a genomic DNA library, or in some other mixture of various DNAs, is conveniently accomplished by the use of an oligonucleotide hybridization probe labelled with a detectable moiety, such as a radioisotope. See Keller et al., DNA Probes, pp.149-213 (Stockton Press, 1989). To identify DNA encoding blocker molecule DNA, the nucleotide sequence of the hybridization probe is preferably selected so that the hybridization probe is capable of hybridizing preferentially to DNA encoding homologues of the equivalent blocker molecule DNA in other species, or variants or derivatives thereof as described herein, under the hybridization conditions chosen. Another method for obtaining blocker molecule is chemical synthesis using one of the methods described, for example, by Engels et al., Agnew. Chem. Int. Ed. Engl. 28:716-734 (1989).

If the entire nucleotide coding sequence for blocker molecule is not obtained in a single cDNA, genomic DNA, or other DNA, as determined, for example, by DNA sequencing or restriction endonuclease analysis, then appropriate DNA fragments (e.g., restriction fragments or PCR amplification products) may be recovered from several DNAs, and covalently joined to one another to construct the entire coding sequence. The preferred means of covalently joining DNA fragments is by ligation using a DNA ligase enzyme, such as T4 DNA ligase.

“Isolated” blocker molecule nucleic acid is blocker molecule nucleic acid that is identified and separated from (or otherwise substantially free from), contaminant nucleic acid encoding other polypeptides. The isolated blocker molecule can be incorporated into a plasmid or expression vector, or can be labeled for probe purposes, using a label as described further herein in the discussion of assays and nucleic acid hybridization methods.

It will be appreciated that if the desired result of the present invention is animals in which female function is repressed, then the blocker molecules may be expressed in vitro, isolated, purified and then delivered to specific organisms. The mode of delivery may be any known procedure including injection and ingestion. Moreover, constructs of the present invention which are capable of expressing blocker molecules may also be delivered by viral vectors like adenovirus.

Site-directed mutagenesis is a preferred method for preparing substitution, deletion, and insertion variants of DNA such as the blocker molecule DNA. This technique is well known in the art; see Zoller et al., Meth. Enz. 100:4668-500 (1983); Zoller, et al., Meth. Enz. 154:329-350 (1987); Carter, Meth. Enz. 154:382-403 (1987); Horwitz et al., Meth. Enz. 185:599-611 (1990), and has been used to produce amino acid sequence variants of trypsin and T4 lysozyme, which variants have certain desired functional properties. Perry et al., Science 226:555-557 (1984); Craik et al., Science 228:291-297 (1985).

Briefly, in carrying out site-directed mutagenesis of blocker molecule DNA, the blocker molecule DNA is altered by first hybridizing an oligonucleotide encoding the desired mutation to a single strand of blocker molecule DNA. After hybridization, a DNA polymerase is used to synthesize an entire second strand, using the hybridized oligonucleotide as a primer, and using the single strand of blocker molecule DNA as a template. Thus the oligonucleotide encoding the desired mutation is incorporated into the resulting double-stranded DNA.

Oligonucleotides for use as hybridization probes or primers may be prepared by any suitable method, such as purification of a naturally occurring DNA or in vitro synthesis. For example, oligonucleotides are readily synthesized using various techniques in such as those described by Narang et al., Meth. Enzymol. 68:90-98 (1979); Brown et al., Meth. Enzymol. 68:109-151 (1979); Caruther et al., Meth. Enzymol. 154:287-313 (1985). The general approach to selecting a suitable hybridization probe or primer is well known. Keller et al., DNA Probes, pp.11-18 (Stockton Press, 1989). Typically, the hybridization probe or primer will contain 10-25 or more nucleotides, and will include at least 5 nucleotides on either side of the sequence encoding the desired mutation so as to ensure that the oligonucleotide will hybridize preferentially to the single-stranded DNA template molecule.

Multiple mutations are introduced into aromatase blocker molecule DNA to produce amino acid sequence variants of aromatase blocker molecule comprising several or a combination of insertions, deletions, or substitutions of amino acid residues as compared to the amino acid-sequences set forth in Figure. If the sites to be mutated are located close together, the mutations may be introduced simultaneously using a single oligonucleotide that encodes all of the desired mutations. If, however, the sites to be mutated are located some distance from each other (separated by more than about ten nucleotides), it is more difficult to generate a single oligonucleotide that encodes all of the desired changes. Instead, one of two alternative methods may be employed.

In the first method, a separate oligonucleotide is generated for each desired mutation. The oligonucleotides are then simultaneously annealed to the single-stranded template DNA, and the second strand of DNA that is synthesized from the template will encode all of the desired amino acid substitutions.

The alternative method involves two or more rounds of mutagenesis to produce the desired mutant. The first round is as described for introducing a single mutation: a single strand of a previously prepared DNA is used as a template, an oligonucleotide encoding the first desired mutation is annealed to this template, and a heteroduplex DNA molecule is then generated. The second round of mutagenesis utilizes the mutated DNA produced in the first round of mutagenesis as the template. Thus this template already contains one or more mutations. The oligonucleotide encoding the additional desired amino acid substitution(s) is then annealed to this template, and the resulting strand of DNA now encodes mutations from both the first and second rounds of mutagenesis. This resultant DNA can be used as a template in a third round of mutagenesis, and so on.

PCR mutagenesis is also suitable for making nucleotide sequence variants of zBMP2 promoter and the blocker molecule. Higuchi, in PCR Protocols, pp.177-183 (Academic Press, 1990); Vallette et al., Nuc. Acids Res. 17:723-733 (1989). Briefly, when small amounts of template DNA are used as starting material in a PCR, primers that differ slightly in sequence from the corresponding region in a template DNA can be used to generate relatively large quantities of a specific DNA fragment that differs from the template sequence only at the positions where the primers differ from the template. For introduction of a mutation into a plasmid DNA, for example, one of the primers is designed to overlap the position of the mutation and to contain the mutation; the sequence of the other primer must be identical to a nucleotide sequence within the opposite strand of the plasmid DNA, but this sequence can be located anywhere along the plasmid DNA. It is preferred, however, that the sequence of the second primer is located within 200 nucleotides from that of the first, such that in the end the entire amplified region of DNA bounded by the primers can be easily sequenced. PCR amplification using a primer pair like the one just described results in a population of DNA fragments that differ at the position of the mutation specified by the primer, and possibly at other positions, as template copying is somewhat error-prone. See Wagner et al., in PCR Topics, pp.69-71 (Springer-Verlag, 1991).

If the ratio of template to product amplified DNA is extremely low, the majority of product DNA fragments incorporate the desired mutation(s). This product DNA is used to replace the corresponding region in the plasmid that served as PCR template using standard recombinant DNA methods. Mutations at separate positions can be introduced simultaneously by either using a mutant second primer, or performing a second PCR with different mutant primers and ligating the two resulting PCR fragments simultaneously to the plasmid fragment in a three (or more)-part ligation.

Another method for preparing variants, cassette mutagenesis, is based on the technique described by Wells et al., Gene, 34:315-323 (1985). The starting material is the plasmid (or other vector) comprising the mAR promoter or blocker molecule DNA to be mutated. The codon(s) in the mAROM promoter or blocker molecule DNA to be mutated are identified. There must be a unique restriction endonuclease site on each side of the identified mutation site(s). If no such restriction sites exist, they may be generated using the above-described oligonucleotide-mediated mutagenesis method to introduce them at appropriate locations in the mAR promoter and blocker molecule DNA. The plasmid DNA is cut at these sites to linearize it. A double-stranded oligonucleotide encoding the sequence of the DNA between the restriction sites but containing the desired mutation(s) is synthesized using standard procedures, wherein the two strands of the oligonucleotide are synthesized separately and then hybridized together using standard techniques. This double-stranded oligonucleotide is referred to as the cassette. This cassette is designed to have 5′ and 3′ ends that are compatible with the ends of the linearized plasmid, such that it can be directly ligated to the plasmid. This plasmid now contains the mutated mAR promoter or blocker molecule DNA sequence.

mAR promoter and blocker molecule DNA, whether cDNA or genomic DNA or a product of in vitro synthesis, is ligated into a replicable vector for further cloning or for expression. “Vectors” are plasmids and other DNAs that are capable of replicating autonomously within a host cell, and as such, are useful for performing two functions in conjunction with compatible host cells (a vector-host system). One function is to facilitate the cloning of the nucleic acid that encodes the mAR promoter and blocker molecule, i.e., to produce usable quantities of the nucleic acid. The other function is to direct the expression of the aromatase blocker molecule. One or both of these functions are performed by the vector-host system. The vectors will contain different components depending upon the function they are to perform as well as the host cell with which they are to be used for cloning or expression.

To produce aromatase blocker molecule, an expression vector will contain nucleic acid that encodes aromatase blocker molecule as described above. The aromatase blocker molecule of this invention may be expressed directly in recombinant cell culture, or as a fusion with a heterologous polypeptide, preferably a signal sequence or other polypeptide having a specific cleavage site at the junction between the heterologous polypeptide and the aromatase blocker molecule.

In one example of recombinant host cell expression, cells are transfected with an expression vector comprising aromatase blocker molecule DNA and the aromatase blocker molecule encoded thereby is recovered from the culture medium in which the recombinant host cells are grown. But the expression vectors and methods disclosed herein are suitable for use over a wide range of prokaryotic and eukaryotic organisms.

Prokaryotes may be used for the initial cloning of DNAs and the construction of the vectors useful in the invention. However, prokaryotes may also be used for expression of mRNA or protein encoded by aromatase blocker molecule. Polypeptides that are produced in prokaryotic host cells typically will be non-glycosylated.

Plasmid or viral vectors containing replication origins and other control sequences that are derived from species compatible with the host cell are used in connection with prokaryotic host cells, for cloning or expression of an isolated DNA. For example, E. coli typically is transformed using pBR322 a plasmid derived from an E. coli species. Bolivar et al., Gene 2:95-113 (1987). PBR322 contains genes for ampicillin and tetracycline resistance so that cells transformed by the plasmid can easily be identified or selected. For it to serve as an expression vector, the pBR322 plasmid, or other plasmid or viral vector, must also contain, or be modified to contain, a promoter that functions in the host cell to provide messenger RNA (mRNA) transcripts of a DNA inserted downstream of the promoter. Rangagwala et al., Bio/Technology 9:477-479 (1991).

In addition to prokaryotes, eukaryotic microbes, such as yeast, may also be used as hosts for the cloning or expression of DNAs useful in the invention. Saccharomyces cerevisiae, or common baker's yeast, is the most commonly used eukaryotic microorganism. Plasmids useful for cloning or expression in yeast cells of a desired DNA are well known, as are various promoters that function in yeast cells to produce mRNA transcripts.

Furthermore, cells derived from multicellular organisms also may be used as hosts for the cloning or expression of DNAs useful in the invention. Mammalian cells are most commonly used, and the procedures for maintaining or propagating such cells in vitro, which procedures are commonly referred to as tissue culture, are well known. Kruse & Patterson, eds., Tissue Culture (Academic Press, 1977). Examples of useful mammalian cells are human cell lines such as 293, HeLa, and WI-38, monkey cell lines such as COS-7 and VERO, and hamster cell lines such as BHK-21 and CHO, all of which are publicly available from the American Type Culture Collection (ATCC), Rockville, Md. 20852 USA.

Expression vectors, unlike cloning vectors, should contain a promoter that is recognized by the host organism and is operably linked to the aromatase blocker molecule nucleic acid. Promoters are untranslated sequences that are located upstream from the start codon of a gene and that control transcription of the gene (that is, the synthesis of mRNA). Promoters typically fall into two classes, inducible and constitutive. Inducible promoters are promoters that initiate high level transcription of the DNA under their control in response to some change in culture conditions, for example, the presence or absence of a nutrient or a change in temperature.

A large number of promoters are known, that may be operably linked to aromatase blocker molecule DNA to block expression of aromatase in a host cell. This is not to say that the promoter associated with naturally occurring aromatase DNA is not usable. However, heterologous promoters generally will result in greater transcription and higher yields of expressed aromtase.

Promoters suitable for use with prokaryotic hosts include the lactamase and lactose promoters, Goeddel et al., Nature 281:544-548 (1979), tryptophan (trp) promoter, Goeddel et al., Nuc. Acids Res. 8:4057-4074 (1980), and hybrid promoters such as the tac promoter, deBoer et al., Proc. Natl. Acad. Sci. USA 80:21-25 (1983). However, other known bacterial promoters are suitable. Their nucleotide sequences have been published, Siebenlist et al., Cell 20:269-281 (1980), thereby enabling a skilled worker operably to ligate them to DNA using linkers or adaptors to supply any required restriction sites. See Wu et al., Meth. Enz. 152:343-349 (1987).

Suitable promoters for use with yeast hosts include the promoters for 3-phosphoglycerate kinase, Hitzeman et al., J. Biol. Chem. 255:12073-12080 (1980); Kingsman et al., Meth. Ehz. 185:329-341 (1990), or other glycolytic enzymes such as enolase, glyceraldehyde-3-phosphate dehydrogenase, hexokinase, pyruvate decarboxylase, phosphofructokinase, glucose-6-phosphate isomerase, 3-phosphoglycerate mutase, pyruvate kinase, triosephosphate isomerase, phosphoglucose isomerase, and glucokinase. Dodson et al., Nuc. Acids res. 10:2625-2637 (1982); Emr, Meth. Enz. 185:231-279 (1990).

Expression vectors useful in mammalian cells typically include a promoter derived from a virus. For example, promoters derived from polyoma virus, adenovirus, cytomegalovirus (CMV), and simian virus 40 (SV40) are commonly used. Further, it is also possible, and often desirable, to utilize promoter or other control sequences associated with a naturally occurring DNA that encodes aromatase, provided that such control sequences are functional in the particular host cell used for recombinant DNA expression.

Other control sequences that are desirable in an expression vector in addition to a promoter are a ribosome binding site, and in the case of an expression vector used with eukaryotic host cells, an enhancer. Enhancers are cis-acting elements of DNA, usually about from 10-300 bp, that act on a promoter to increase the level of transcription. Many enhancer sequences are now known from mammalian genes (for example, the genes for globin, elastase, albumin, α-fetoprotein and insulin). Typically, however, the enhancer used will be one from a eukaryotic cell virus. Examples include the SV40 enhancer on the late side of the replication origin (bp 100-270), the cytomegalovirus early promoter enhancer, the polyoma enhancer on the late side of the replication origin, and adenovirus enhancers. See Kriegler, Meth. Enz. 185:512-527 (1990).

Expression vectors may also contain sequences necessary for the termination of transcription and for stabilizing the messenger RNA (mRNA). Balbas et al., Meth. Enz. 185:14-37 (1990); Levinson, Meth. Enz. 185:485-511 (1990). In the case of expression vectors used with eukaryotic host cells, such transcription termination sequences may be obtained from the untranslated regions of eukaryotic or viral DNAs or cDNAs. These regions contain polyadenylation sites as well as transcription termination sites. Birnsteil et al., Cell 41:349-359 (1985).

In general, control sequences are DNA sequences necessary for the expression of an operably linked coding sequence in a particular host cell. “Expression” refers to transcription and/or translation. “Operably linked” refers to the covalent joining of two or more DNA sequences, by means of enzymatic ligation or otherwise, in a configuration relative to one another such that the normal function of the sequences can be performed. For example, DNA for a pre-sequence or secretory leader is operably linked to DNA for a polypeptide if it is expressed as a preprotein that participates in the secretion of the polypeptide; a promoter or enhancer is operably linked to a coding sequence if it affects the transcription of the sequence; or a ribosome binding site is operably linked to a coding sequence if it is positioned so as to facilitate translation. Generally, “operably linked” means that the DNA sequences being linked are contiguous and, in the case of a secretory leader, contiguous and in reading frame. Linking is accomplished by ligation at convenient restriction sites. If such sites do not exist, then synthetic oligonucleotide adaptors or linkers are used, in conjunction with standard recombinant DNA methods.

Expression and cloning vectors also will contain a sequence that enables the vector to replicate in one or more selected host cells. Generally, in cloning vectors this sequence is one that enables the vector to replicate independently of the host chromosome(s), and includes origins of replication or autonomously replicating sequences. Such sequences are well known for a variety of bacteria, yeast, and viruses. The origin of replication from the plasmid pBR322 is suitable for most gram-negative bacteria, the 2μ plasmid origin is suitable for yeast, and various viral origins (for example, from SV40, polyoma, or adenovirus) are useful for cloning vectors in mammalian cells. Most expression vectors are “shuttle” vectors, i.e. they are capable of replication in at least one class of organisms but can be transfected into another organism for expression. For example, a vector may be cloned in E. coli and then the same vector is transfected into yeast or mammalian cells for expression even though it is not capable of replicating independently of the host cell chromosome.

The expression vector may also include an amplifiable gene, such as that comprising the coding sequence for dihydrofolate reductase (DHFR). Cells containing an expression vector that includes a DHFR gene may be cultured in the presence of methotrexate, a competitive antagonist of DHFR. This leads to the synthesis of multiple copies of the DHFR gene and, concomitantly, multiple copies of other DNA sequences comprising the expression vector, Ringold et al., J. mol. Apl. Genet. 1:165-175 (1981), such as a DNA sequence encoding the aromatase blocker molecule. In that manner, the level of the aromatase blocker molecule produced by the cells may be increased.

DHFR protein encoded by the expression vector also may be used as a selectable marker of successful transfection. For example, if the host cell prior to transformation is lacking in DHFR activity, successful transformation by an expression vector comprising DNA sequences encoding the aromatase blocker molecule and DHFR protein can be determined by cell growth in medium containing methotrexate. Also, mammalian cells transformed by an expression vector comprising DNA sequences encoding the aromatase blocker molecule, DHFR protein, and aminoglycoside 3′ phosphotransferase (APH) can be determined by cell growth in medium containing an aminoglycoside antibiotic such as kanamycin or neomycin. Because eukaryotic cells do not normally express an endogenous APH activity, genes encoding APH protein, commonly referred to as neo^{r} genes, may be used as dominant selectable markers in a wide range of eukaryotic host cells, by which cells transfected-by the vector can easily be identified or selected. Jiminez et al., Nature, 287:869-871 (1980); Colbere-Garapin et al., J. Mol. Biol. 150:1-14 (1981); Okayama & Berg, Mol. Cell. Biol., 3:280-289 (1983).

Many other selectable markers are known that may be used for identifying and isolating recombinant host cells that express the aromatase blocker molecule. For example, a suitable selection marker for use in yeast is the trp1 gene present in the yeast plasmid YRp7. Stinchcomb et al., Nature 282:39-43 (1979); Kingsman et al., Gene 7:141-152 (1979); Tschemper et al., Gene 10:157-166 (1980). The trp1 gene provides a selection marker for a mutant strain of yeast lacking the ability to grow in tryptophan, for example, ATCC No. 44076 or PEP4-1 (available from the American Type Culture Collection, Rockville, Md. 20852 USA). Jones, Genetics 85:12 (1977). The presence of the trp1 lesion in the yeast host cell genome then provides an effective environment for detecting transformation by growth in the absence of tryptophan. Similarly, Leu2-deficient yeast strains (ATCC Nos. 20622 or 38626) are complemented by known plasmids bearing the Leu2 gene.

Particularly useful in the invention are expression vectors that provide for the transient expression of DNA encoding the aromatase blocker molecule. In general, transient expression involves the use of an expression vector that is able to efficiently replicate in a host cell, such that the host cell accumulates many copies of the expression vector and, in turn, synthesizes high levels of a desired polypeptide encoded by the expression vector. Transient expression systems, comprising a suitable expression vector and a host cell, allow for the convenient positive identification of polypeptides encoded by cloned DNAs, as well as for the rapid screening of such polypeptides for desired biological or physiological properties. Yang et al., Cell 47:3-10 (1986); Wong et al., Science 228:810-815 (1985); Lee et al., Proc. Nat Acad. Sci. USA 82:4360-4364 (1985). Thus, transient expression systems are particularly useful in the invention for expressing DNAs encoding amino acid sequence variants of the aromatase blocker molecule, to identify those variants which are functionally active.

Since it is often difficult to predict in advance the characteristics of an amino acid sequence variant of the aromatase blocker molecule, it will be appreciated that some screening of such variants will be needed to identify those that are functionally active. Such screening may be performed in vitro, using routine assays for receptor binding, or assays for cell proliferation, cell differentiation or cell viability, or using immunoassays with monoclonal antibodies that selectively bind to the aromatase blocker molecule that effect the functionally active aromatase blocker molecule, such as a monoclonal antibody that selectively binds to the active site or receptor binding site of the aromatase blocker molecule.

As used herein, the terms “transformation” and “transfection” refer to the process of introducing a desired nucleic acid, such a plasmid or an expression vector, into a host cell. Various methods of transformation and transfection are available, depending on the nature of the host cell. In the case of E. coli cells, the most common methods involve treating the cells with aqueous solutions of calcium chloride and other salts. In the case of mammalian cells, the most common methods are transfection mediated by either calcium phosphate or DEAE-dextran, or electroporation. Sambrook et al., eds., Molecular Cloning, pp. 1.74-1.84 and 16.30-16.55 (Cold Spring Harbor Laboratory Press, 1989). Following transformation or transfection, the desired nucleic acid may integrate into the host cell genome, or may exist as an extrachromosomal element.

Host cells that are transformed or transfected with the above-described plasmids and expression vectors are cultured in conventional nutrient media modified as is appropriate for inducing promoters or selecting for drug resistance or some other selectable marker or phenotype. The culture conditions, such as temperature, pH, and the like, suitably are those previously used for culturing the host cell used for cloning or expression, as the case may be, and will be apparent to those skilled in the art.

Suitable host cells for cloning or expressing the vectors herein are prokaryotes, yeasts, and higher eukaryotes, including insect, oysters, lower vertebrate, and mammalian host cells. Suitable prokaryotes include eubacteria, such as Gram-negative or Gram-positive organisms, for example, E. coli, Bacillus species such as B. subtilis, Pseudomonas species such as P. aeruginosa, Salmonella typhimurium, or Serratia marcescans.

In addition to prokaryotes, eukaryotic microbes such as filamentous fungi or yeast are suitable hosts for blocker molecule-encoding vectors. Saccharomyces cerevisiae, or common baker's yeast, is the most commonly used among lower eukaryotic host microorganisms. However, a number of other genera, species, and strains are commonly available and useful herein, such as Schizosaccharomyces pombe, Beach and Nurse, Nature 290:140-142 (1981), Pichia pastoris, Cregg et al., Bio/Technology 5:479-485 (1987); Sreekrishna, et al., Biochemistry 28:4117-4125 (1989), Neurospora crassa, Case, et al., Proc. Natl. Acad. Sci. USA 76:5259-5263 (1979), and Aspergillus hosts such as A. nidulans, Ballance et al., Biochem. Biophys. Res. Commun. 112:284-289 (1983); Tilburn et al., Gene 26:205-221 (1983); Yelton et al., Proc. Natl. Acad. Sci. USA 81:1470-1474 (1984), and A. niger, Kelly et al., EMBO J. 4:475-479 (1985).

Suitable host cells for the expression of the aromatase blocker molecule also are derived from multicellular organisms. Such host cells are capable of complex processing and glycosylation activities. In principle, any higher eukaryotic cell culture is useable, whether from vertebrate or invertebrate culture. It will be appreciated, however, that because of the species-, tissue-, and cell-specificity of glycosylation, Rademacher et al., Ann. Rev. Biochem. 57:785-838 (1988), the extent or pattern of glycosylation of HoxCG in a foreign host cell typically will differ from that of the aromatase blocker molecule obtained from a cell in which it is naturally expressed.

Examples of invertebrate cells include insect and plant cells. Numerous baculoviral strains and variants and corresponding permissive insect host cells from hosts such as Spodoptera frugiperda (caterpillar), Aedes aegypti (mosquito), Aedes albopictus (mosquito), Drosophila melanogaster (fruitfly), and Bombyx mori host cells have been identified. Luckow et al., Bio/Technology 6:47-55 (1988); Miller et al., in Genetic Engineering, vol. 8, pp.277-279 (Plenum Publishing, 1986); Maeda et al., Nature 315:592-594 (1985).

Plant cell cultures of cotton, corn, potato, soybean, petunia, tomato, and tobacco can be utilized as hosts. Typically, plant cells are transfected by incubation with certain strains of the bacterium Agrobacterium tumefaciens. During incubation of the plant cells with A. tumefaciens, the DNA is transferred into cells, such that they become transfected, and will, under appropriate conditions, express the introduced DNA. In addition, regulatory and signal sequences compatible with plant cells are available, such as the nopaline synthase promoter and polyadenylation signal sequences, and the ribulose biphosphate carboxylase promoter. Depicker et al., J. Mol. Appl. Gen. 1:561-573 (1982). Herrera-Estrella et al., Nature 310:115-120 (1984). In addition, DNA segments isolated from the upstream region of the T-DNA 780 gene are capable of activating or increasing transcription levels of plant-expressible genes in recombinant DNA-containing plant tissue. European Pat. Pub. No. EP 321,196 (published Jun. 21, 1989).

However, interest has been greatest in vertebrate cells, and propagation of vertebrate cells in culture (tissue culture) has become a routine procedure in recent years. Kruse & Patterson, eds., Tissue Culture (Academic Press, 1973). Examples of useful mammalian host cells are the monkey kidney CV1 line transformed by SV40 (COS-7, ATCC CRL 1651); human embryonic kidney line 293 (or 293 cells subcloned for growth in suspension culture), Graham et al., J. Gen Virol. 36:59-72 (1977); baby hamster kidney cells (BHK, ATCC CCL 10); Chinese hamster ovary cells (including DHFR-deficient CHO cells, Urlaub et al., Proc. Natl. Acad. Sci. USA 77:4216-4220 (1980); mouse sertoli cells (TM4, Mather, Biol. Reprod. 23:243-251 (1980); monkey kidney cells (CV1, ATCC CCL 70); African green monkey kidney cells (VERO-76, ATCC CRL-1587); human cervical carcinoma cells (HELA, ATCC CCL 2); canine kidney cells (MDCK, ATCC CCL 34); buffalo rat liver cells (BRL 3A, ATCC CRL 1442); human lung cells (W138, ATCC CCL 75); human liver cells (Hep G2, HB 8065); mouse mammary tumor (MMT 060562, ATCC CCL51); TRI cells (Mather et al., Annals N.Y. Acad. Sci. 383:44-68 (1982)); MRC 5 cells; FS4 cells; and a human hepatoma line (Hep G2).

Construction of suitable vectors containing the nucleotide sequence encoding the aromatase blocker molecule and appropriate control sequences employs standard recombinant DNA methods. DNA is cleaved into fragments, tailored, and ligated together in the form desired to generate the vectors required.

For analysis to confirm correct sequences in the vectors constructed, the vectors are analyzed by restriction digestion (to confirm the presence in the vector of predicted restriction endonuclease) and/or by sequencing by the dideoxy chain termination method of Sanger et al., Proc. Nat. Acad. Sci. USA 72:3918-3921 (1979).

The cells used to produce the aromatase blocker molecule of this invention may be cultured in a variety of media. Commercially available media such as Ham's F10 (Sigma), Minimal Essential Medium (MEM, Sigma), RPMI-1640 (Sigma), and Dulbecco's Modified Eagle's Medium (DMEM, Sigma) are suitable for culturing the host cells. In Iaddition, any of the media described in Ham, et al., Meth. Enz. 58:44-93 (1979); Barnes et al., Anal. Biochem. 102:255-270 (1980); Bottenstein et al., Meth. Enz. 58:94-109 (1979); U.S. Pat. Nos. 4,560,655; 4,657,866; 4,767,704; or 4,927,762; or in PCT Pat. Pub. Nos. WO 90/03430 (published Apr. 5, 1990), may be used as culture media for the host cells. Any of these media may be supplemented as necessary with hormones and/or other growth factors (such as insulin, transferrin, or epidermal growth factor), salts (such as sodium chloride, calcium, magnesium, and phosphate), buffers (such as HEPES), nucleosides (such as adenosine and thymidine), antibiotics, trace elements (defined as inorganic compounds usually present at final concentrations in the micromolar range), and glucose or an equivalent energy source. Any other necessary supplements may also be included at appropriate concentrations that would be known to those skilled in the art. The culture conditions, such as temperature, pH, and the like, are those previously used with the host cell selected for expression, and will be apparent to the ordinarily skilled artisan.

The host cells referred to in this disclosure encompass cells in culture in vitro as well as cells that are within a host animal, for example, as a result of transplantation or implantation.

It is further contemplated that the aromatase blocker molecule of this invention may be produced by homologous recombination, for example, as described in PCT Pat. Pub. No. WO 91/06667 (published May 16, 1991). Briefly, this method involves transforming cells containing an endogenous gene encoding aromatase blocker molecule with a homologous DNA, which homologous DNA comprises (1) an amplifiable gene, such as DHFR, and (2) at least one flanking sequence, having a length of at least about 150 base pairs, which is homologous with a nucleotide sequence in the cell genome that is within or in proximity to the gene encoding aromatase blocker molecule. The transformation is carried out under conditions such that the homologous DNA integrates into the cell genome by recombination. Cells having integrated the homologous DNA then are subjected to conditions which select for amplification of the amplifiable gene, whereby the aromatase blocker molecule gene amplified concomitantly. The resulting cells then are screened for production of desired amounts of aromatase blocker molecule. Flanking sequences that are in proximity to a gene encoding aromatase blocker molecule are readily identified, for example, by the method of genomic walking, using as a starting point the aromatase blocker molecule nucleotide sequences set forth in SEQ ID NO.:8 and SEQ ID NO.1:3. See Spoerel et al., Meth. Enz. 152:598-603 (1987).

Gene amplification and/or gene expression may be measured in a sample directly, for example, by conventional Southern blotting to quantitate DNA, or Northern blotting to quantitate mRNA, using an appropriately labeled oligonucleotide hybridization probe, based on the sequences provided herein. Various labels may be employed, most commonly radioisotopes, particularly ³²P. However, other techniques may also be employed, such as using biotin-modified nucleotides for introduction into a polynucleotide. The biotin then serves as the site for binding to avidin or antibodies, which may be labeled with a wide variety of labels, such as radioisotopes, fluorophores, chromophores, or the like. Alternatively, antibodies may be employed that can recognize specific duplexes, including DNA duplexes, RNA duplexes, and DNA-RNA hybrid duplexes or DNA-protein duplexes. The antibodies in turn may be labeled and the assay may be. carried out where the duplex is bound to a surface, so that upon the formation of duplex on the surface, the presence of antibody bound to the duplex can be detected.

Gene expression, alternatively, may be measured by immunological methods, such as immunohistochemical staining of tissue sections and assay of cell culture or body fluids, to quantitate directly the expression of the gene product. With immunohistochemical staining techniques, a cell sample is prepared, typically by dehydration and fixation, followed by reaction with labeled antibodies specific for the gene product coupled, where the labels are usually visually detectable, such as enzymatic labels, fluorescent labels, luminescent labels, and the like. A particularly sensitive staining technique suitable for use in the present invention is described by Hsu et al., Am. J. Clin. Path., 75:734-738 (1980). Antibodies useful for immunohistochemical staining and/or assay of sample fluids may be either monoclonal or polyclonal. Conveniently, the antibodies may be prepared against a synthetic peptide based on the DNA sequences provided herein.

Throughout the description and claims of this specification, the word “comprise” and variations of the word, such as “comprising” and “comprises”, means “including but not limited to” and is not intended to exclude other additives, components, integers or steps.

The invention will now be further described by way of reference only to the following non-limiting examples. It should be understood, however, that the examples following are illustrative only, and should not be taken in any way as a restriction on the generality of the invention described above. Amino acid sequences referred to herein are given in standard single letter code.

### EXAMPLE 1

### Evaluation of Effectiveness of a Sex Differentiating Construct for Controlling the Abundance of Pest Animals. A. Mendelian Genetic Model

To determine the effectiveness of the invention to affect the sex ratio of pest populations, and thereby the pest abundance, the applicants used Mendelian models to assess the effect of an introduced sexdifferentiation construct (SDC), i.e., a construct that determines phenotypic sex. The model assumes that the construct is stable and integrated into an chromosome, that one copy of the SDC causes maleness, irrespective of genotypic sex, and that transgenic fish are rare in the population, and hence all crosses are to wild type females.

Introduction of males homozygous for an SDC on any chromosome pair results in 100% males being produced in the F1 generation. The same is true for parental fish in which the SDC is not integrated chromosomally, but transmited to the next generation as plasmids, contingent on expression patterns.

Second, the sex ratio of subsequent generations depends on the number of SDCs in the F1 individuals. Specifically, assuming random assortment of chromosomes in all matings, the distribution of the number of chromosomes carrying an SDC in generation K+1 is a binomial distribution of the form (a+b)^{N} (where N equals the number of chromosomes carrying an SDC in each male in generation K and a and b equal 1) times the number of males carrying N=0,1,2,3,4. . . Nₘₐₓ in generation K. Hence the ratio of offspring type for a male with N=4 is 1 offspring with 0 SDCs (and hence female), 4 carrying 1 copy, 6 carrying 2, 4 carrying 3, and 1 carrying 4 copies, producing a sex ratio of 15/1 (94% male). In the subsequent generation, the four males carrying one copy of the SDC would produce a 50:50 sex ratio, the six carrying 2 copies would produce offspring with a ratio of 1:2:1 of N=0,1 and 2, respectively, the four carrying 3 copies would produce offspring at a ratio of 1:3:3:1 of N=0,1,2,and 3, respectively, and the one male carrying four copies produces a ratio of 1:4:6:4:1 of N=0,1,2,3,and 4, respectively. The summed distribution of SDC numbers in the second generation is 15:32:10:5:1, for N=0,1,2,3 and 4, respectively, and a sex ratio of 48/15 (76% male).

Third, as all matings are with wild-type females (N=0), mean N declines each generation, approximately as a factor of 2. The endpoint is a mean less than 1, at which point subsequent generations have a sex ratio only marginally higher than 50:50 due to the predominating effect of the natural sex chromosomes. However, because the distribution of N is truncated at zero, the decline in sex ratio with each generation to 50:50 is slow and approaches 50:50 assymptotically.

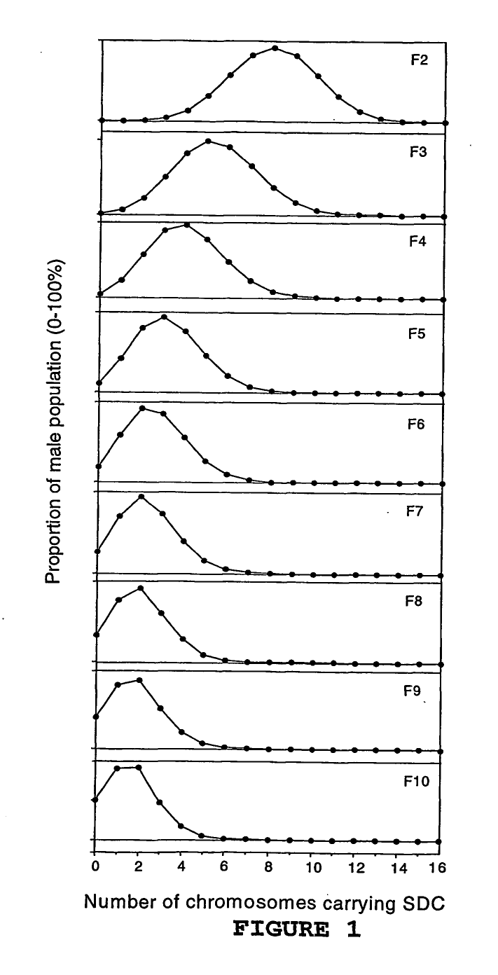

The effect of the asymmetric distribution of N about the population mean in each generation is illustrated in FIG. 1. The stocked individuals are assumed to have 32 chromosomes (including the two sex chromosomes), and have SDCs on all 32, i.e., they are homozygous on all 16 chromosome pairs for SDCs. All matings are with wild-type females. F1 is 100% male, as all offspring carry 16 SDCs. As expected, the mean number of SDC-bearing chromosomes in each subsequent generation declines to approximately 8 in F3, and slightly more than 4 in F4. Thereafter, however, the decline to a mean of 1 is slowed dramatically due to the skew to the left and the elimination of N=0 individuals (because they are female) from subsequent generations.

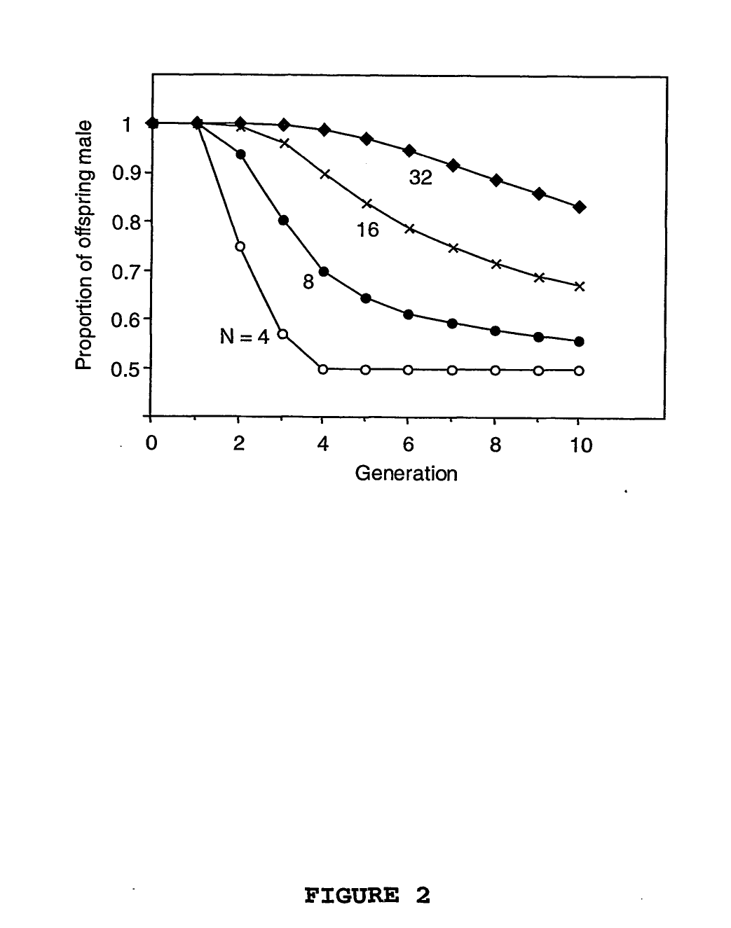

The effect on sex ratios is indicated in FIG. 2, which compares the decline in proportion of males produced over subsequent generations for stocked individuals carrying 2, 4, 8 and 16 homozygous pairs (N=4, 8, 16 and 32, respectively). At the highest N examined (32), the males exceed 99% of all offspring produced through to F4, exceeds 90% male through to F7, and by F10 is still 84% male biased. For species in which chromosome numbers are high (e.g., carp), loading multiple copies of the SDC onto most or all chromosomes results in an effective ‘infinite’ copy number, relative to the number of generations required to cause total eradication, and male biases at or close to 100% male for greater than 10 generations.

### EXAMPLE 2

### Evaluation of Effectiveness of a Sex Differentiation Construct for Controlling the Abundance of Pest Animals. B. Population Model

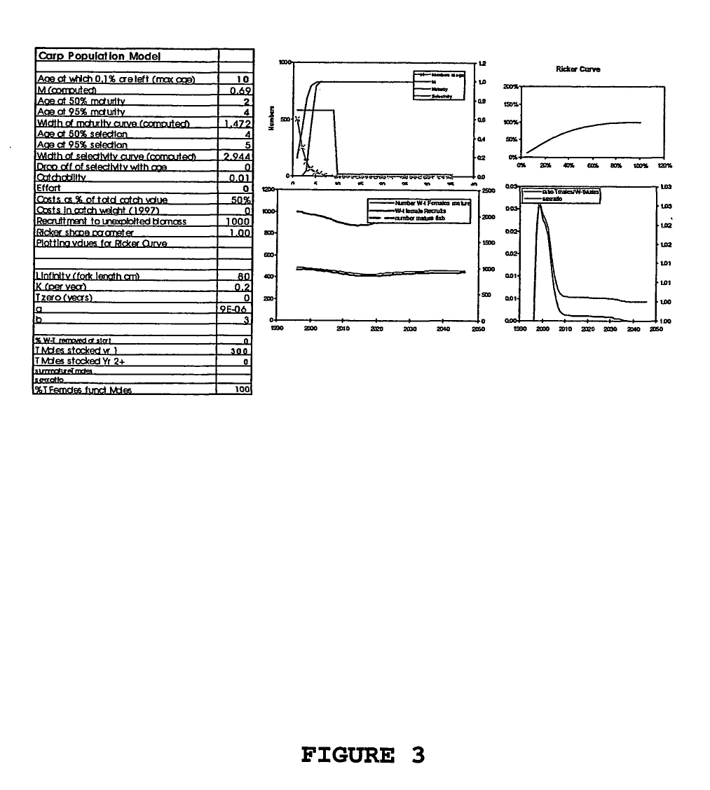

In order to assess the effect of the introduction of a sex determining construct on the viability of pest populations, the inventors developed and evaluated the performance of the construct for a realistic population model for a well-known pest (carp, Cyprinus carpio). The model was implemented using Excel (FIG. 3), and incorporated the following features:

The starting number of reproductive females (at to) was determined by the carrying capacity of juveniles as subsequently modified by rates of natural mortality or harvesting.

At t₀ the sex ratio is 50:50.

The environmental carrying capacity was 1000 juveniles.

The natural (un-augmented) number of new recruits each year was determined based on the number of adults relative to carrying capacity via a Ricker stock-recruitment relationship. The shape of the Ricker curve could be modified by varying the two principal parameters.

For illustrative purposes, in initial runs it was assumed that there was no year-to-year variability in recruitment and mortality caused by environmental variability.

Age at sexual maturity was set at 2 years.

Mortality was age-specific, and could be varied independently for the juvenile and adult life history stages. Age at 99% mortality was set at 10 years.

Fishing mortality could be varied independently of natural mortality, and was variably size (age) dependent.

Individuals carrying SDCs are stocked as recruits.

The effect of an SDC could be set to either sterilise female genotypes or convert them into functional males.

For purposes of the model, the effect of an SDC was to produce 100% males for N generations following stocking, where N could be varied from 1 to 5. At N+1, the sex ratio of the offspring dropped immediately to 50:50. Based on Example 1, this is a conservative estimate of the effect on population sex ratio of stocking individuals with a high number of chromosomes carrying SDCs. Hence the model is conservative.

The general parameter values specified for the model are given in FIG. 3, and are best estimates for carp, taken as a model species. This model was run under a number of different scenarios to examine the combined effects of different stocking regimes, different SDC copy numbers in stocked fish (=number of generations before the sex ratio reverted to the 1:1 wild type), effects of harvesting, effects of environmental variability of model performance and the effect of a SDC as opposed to a construct that has only sterilises females (rather than causing them to become functional males).

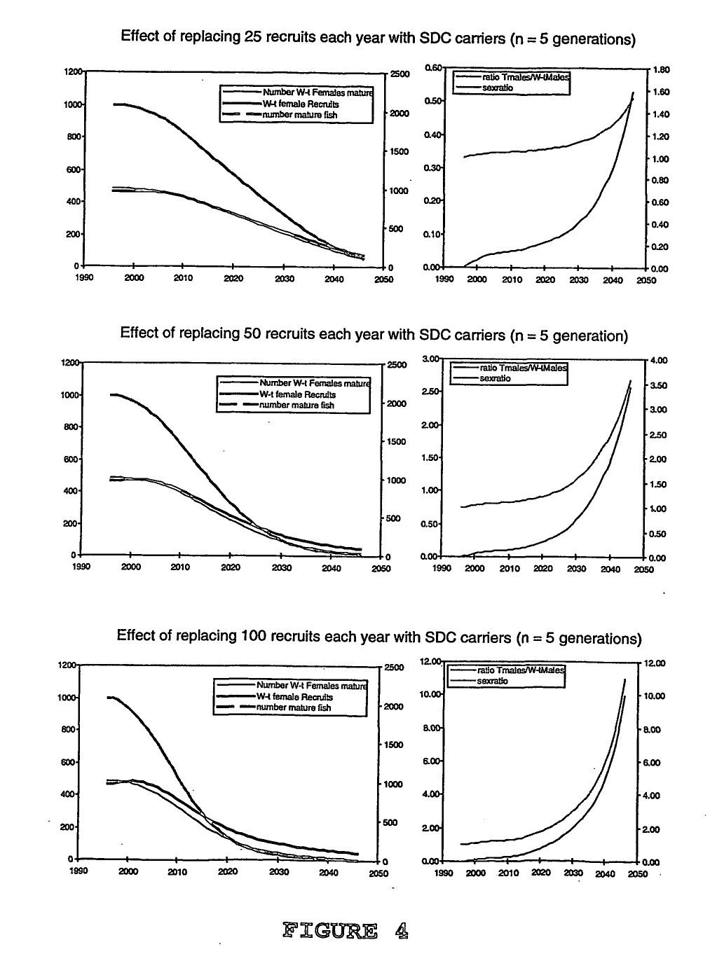

Principal model outcomes are illustrated in FIGS. 4 to 8.

The effect of adding 25 (2.5% of natural recruitment), 150 (5%) and 100 (10%) juveniles carrying N=5 generations copies of the SDC annually, on population sex ratio, recruitment and adult population numbers for the subsequent 50 years, is shown in FIG. 4. Introduction of the SDC-carrying juveniles causes a long-term decline in natural recruitment of the targeted population, the rate of which increases as the number of stocked fish increases. This effect is due to the declining number of females remaining in the population, with the consequent decline in recruitment buffered for about ten years by the non-linearity of the Ricker stock-recruitment relationship. Linearising the stock-recruitment relationship results in an immediate and steep decline in natural recruitment and adult numbers (results of simulation not shown). Annual stocking of 50 SDC-carrying juveniles, under best-estimated biological parameters of a natural carp population and ignoring the inevitable complications of spatial dynamics and natural variability in recruitment and adult mortality (see discussion below), results in extinction of the target population in about 50 years from release. Increasing the number of SDC-carrying juveniles released each year to 100 (10% of natural recruitment) reduces the amount of time to extinction of the targeted species.

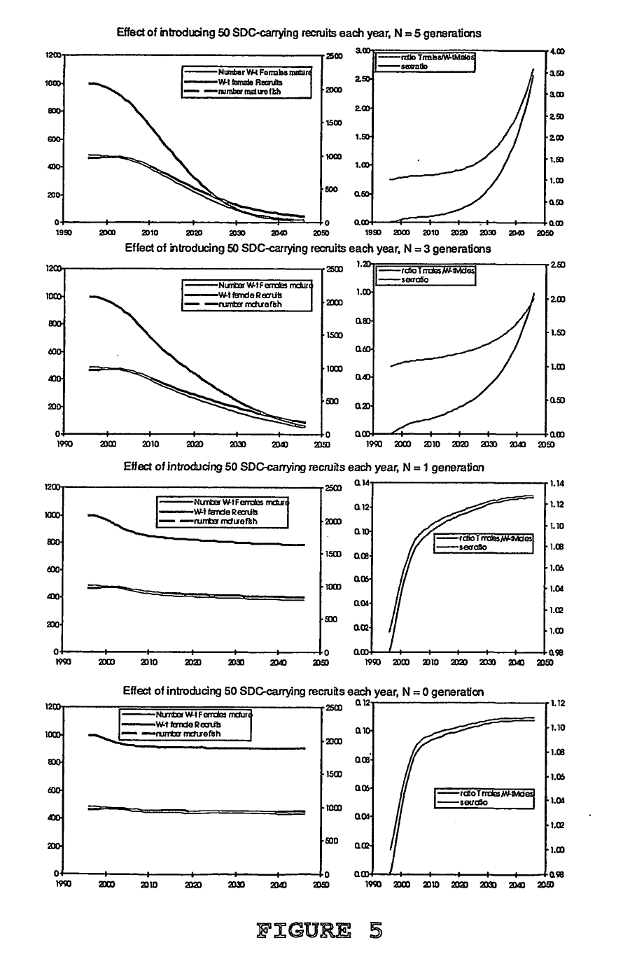

The rate and magnitude of the decline in target population size varies with SDC copy number (FIG. 5), as expected based on the effects of copy number on sex ratios in each subsequent generation. At the lowest level, if the hypothesised effect of an SDC is limited to only the stocked generation (N=0), then annual stocking at 5% of natural recruitment (e.g., 50 SDC-carrying juveniles) depresses natural recruitment cumulatively by about 7%, produces a long-term shift in population sex ratio to about 1.1 males per female, and has a negligible effect on population size (due to buffering effects of the non-linear stock-recruitment relationship). Increasing the SDC copy number to achieve all male offspring in F1 (hence N=1 generation), results in a slow decline in recruitment to about 80% of virgin after 50 years, a shift in population Sex ratio only slightly higher than that caused by the N=0 generation SDC, and a long, slow decline in parental-biomass. The effects of stocking SDC-carrying fish are much more pronounced at N=3 and N=5 generations. The cumulative effect of sex ratios that are heavily biased towards males for 3 and 5 generations after release, respectively, results in an exponential increase in the number of males in each generation, and a consequent steep increase in population sex ratios and rapid rates of decline in natural recruitment and adult population numbers.

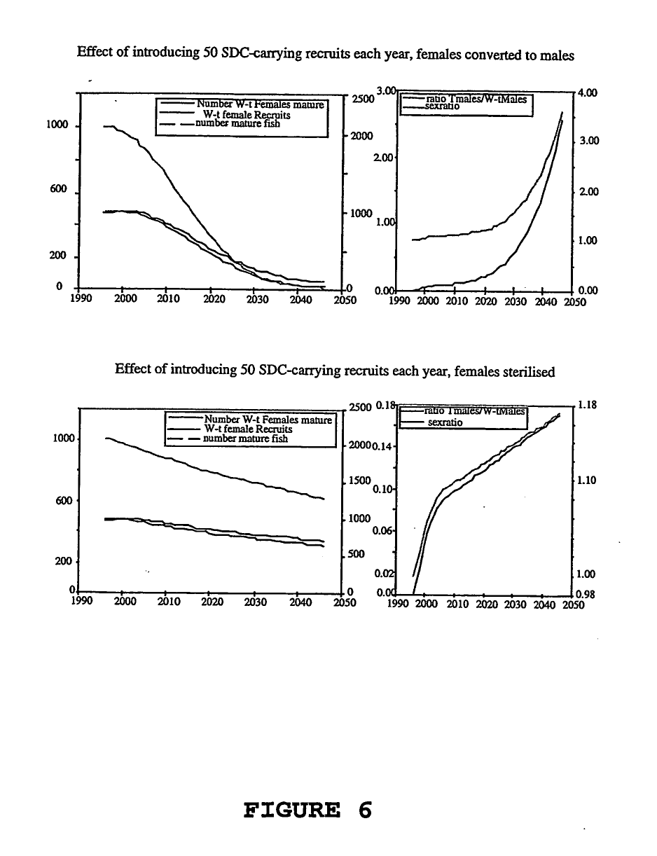

The multiplicative effect of high copy numbers is also indicated by comparing the effects of an SDC at N=5 generations with an equivalent construct that does not convert genetic females to functional males, but which only sterilises the females (FIG. 6). Annual stockings of 50 juveniles results in a 90+ percent decline in number of adults over a 50 year period, when the SDC converts females to males, but only about a 30% decline when the construct only sterilises females. The end point of the stocking program in both cases is extinction of the target population, but the rate of decline is greatly increased due to the cumulative effect of increasing the number of carrier males in the population generated by converting females to carrier males. Nonetheless, pest control can also be accomplished by a construct that results in sex-linked sterility.

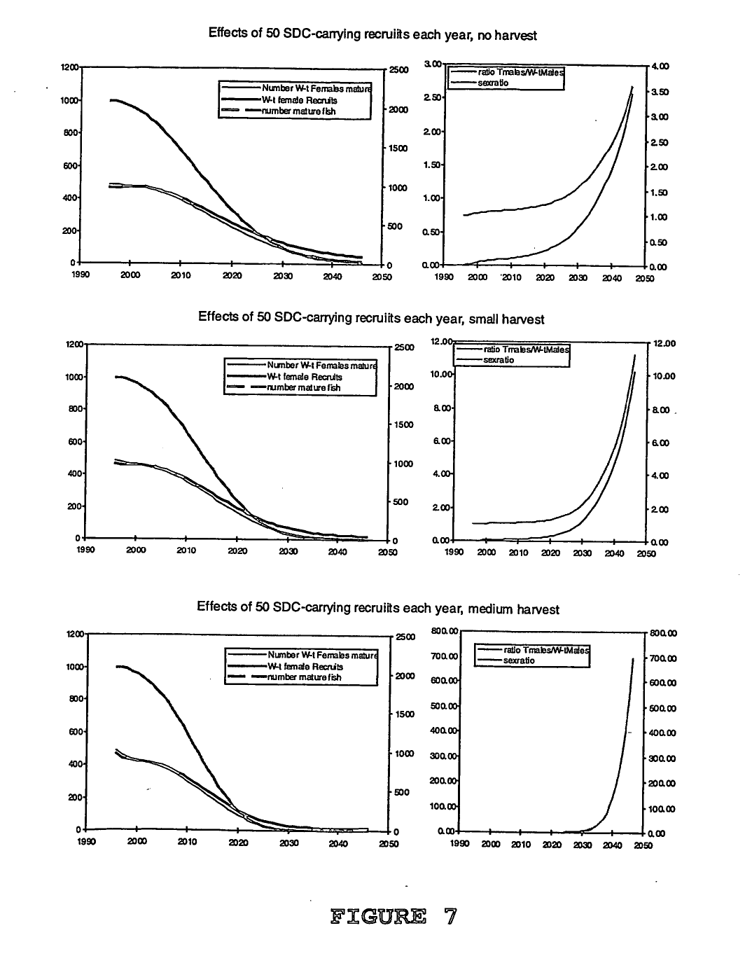

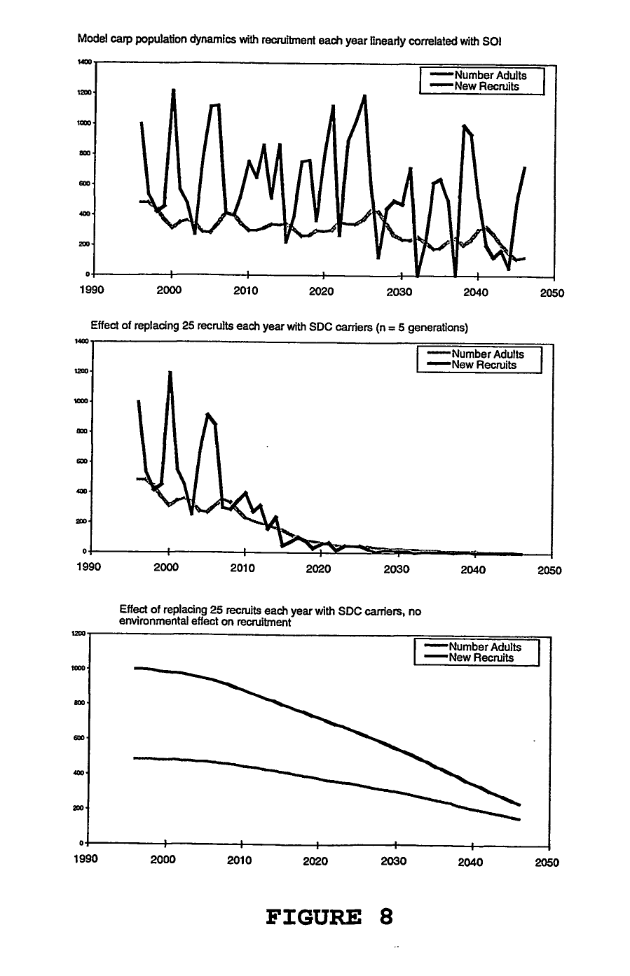

The rate of population decline can also be accelerated by harvesting or otherwise removing wild-type males and females from the target population (FIG. 7). Adding a small annual harvest (approximately 1.4% annual removal of adults)(by, for example, selective fishing targeting large adults) reduces time to extinction from by more than 10 years in the modelled system; increasing the fishing pressure to a ‘moderate’ level (approximately 5.3% annual removal) reduces time to extinction by a further 10-15 years.

The effects of annual harvests are similar to that which would be expected from highly variable rates of natural adult mortality, as caused by, for example, droughts. Any factor that increases the rate at which wild-type adults are removed from the system speeds up the rate of extinction by reducing the target population's ability to buffer the effect of declining female numbers due to the stock-recruitment relationship. Similarly, the rate of extinction also depends on the number of stocked SDC-carrying juveniles relative to rates of natural recruitment. This is illustrated in FIG. 8, in which natural recruitment is allowed to vary as a linear function of the Southern Oscillation Index (dry years=poor recruitment, which is consistent with field observations). Stocking rates can be held constant due to artificial breeding programs, whereas the natural recruitment varies depending on environmental conditions. Hence, a stocking rate of 5% of natural recruitment for an good recruitment year could increase to >1000% in years of exceptionally poor natural recruitment, the effect of which is to steeply increase the rate of reproductive success of SDC-carrying males and the consequent rate of female population decline.

In nature, the rate of population decline will also depend on spatial dynamics, as these affect rate of gene flow and the spread of the SDC. Stocking regimes can be optimised to maximise rate of spread. The effect of restricted gene flow between populations on long-term viability depends on the rate of genetic exchange and SDC copy number. When copy number is low and gene flow is low, the effect on peripheral populations will be slight; when either or both values are high, peripheral populations will be subject to nearly the same rate of extinction as the stocked population. Because of the interaction between copy number and gene flow on rates of population decline, copy number in stocked animals could be adjusted as to minimise effects of stocking on peripheral populations, if desired, while still maximising impacts on the target population. An example where this might be-desirable is the use of an SDC to control invasive lamprey populations in the North American Great Lakes, in which low copy number individuals stocked into the Great Lakes could be used to reduce and possibly eradicate lampreys locally, while minimising the risk to the native lamprey population in the North Atlantic.

### EXAMPLE 3

### The Role of Aromatse in Sex Differentiation in Animals

Aromatase is the enzyme that in all vertebrates examined converts C19 steroids (androgens) to C18 steroids (estrogens)(Ozon, 1972). Specifically, aromatase acts on bipotential embryonic or juvenile gonads, which are male by default in vertebrates, to convert the male hormones (androgens) to females hormones (estrogens) and shift gonad development into the ovarian pathway. Aromatase has also been implicated as the key enzyme in sex determination in a number of invertebrate groups, including gastropods (Oberdoster, 1997; Mattjiessen & Gibbs, 1998), bivalves (Matsumoto, et al., 1997), and echinoderms (Hines, et al., 1992).

Aromatase activity can be inhibited chemically, by the addition in water (for aquatic animals) or diet of specific and non-reversible inhibitors (specifically 4-hydroxyandrostenedione and 1,4,6-androstatriene-3-17-dione)(see references in table 1). In lower vertebrates (fish, amphibians and reptiles), aromotase inhibition causes partial or complete masculinisation of genetic females, producing phenotypic sex bias in broods of up to 100% male. Masculinised females, in such lower vertebrates, are fully functional, fertile and apparently normal males (Piferrer, et al., 1994) despite their genetic sex. Brief treatment with an aromatase inhibitor is a standard method used by aquaculturists to produce broods that are predominantly male (males are more valuable than females, as they do not invest energy in producing large gonads). In higher vertebrates (birds), aromatase inhibition results in female sterilisation, due to disruption of normal female gonadogenesis (Shimada, et al., 1996). A similar effect has been demonstrated in molluscs (snails) by Bettin, et al. (1996). Whether aromatase inhibition in snails results in functional males is not known, though in at least one species high levels of tributyl tin contamination (which is thought to competitively inhibit aromatisation) resulted in at least the development of early spermatogenic tissues (Gibbs, et al., 1988). In mammals, aromatase is involved in a number of physiological activities, and its inhibition would result in widespread physiological dysfunction.

Sensitivity of phenotypic sex to aromotase inhibition is stage specific, due to the developmental sequence of gonadagenesis. Piferrer, et al., (1994) demonstrate that treatment of embryonic fish (salmon) for as little as two hours with a water soluable aromatase inhibitor, in this case 3 days after 50% hatching from eggs, results in male biases as high as 98%.

TABLE 1
SPECIES IN WHICH AROMATASE INHIBITION HAS BEEN
DEMONSTRATED TO AFFECT PHENOTYPIC SEX

| ANIMAL | SPECIES | REFERENCE |
|---|---|---|
| Fish: | | |
| Salmon | Onchorhychus | Piferrer et al. (1994) |
| | tshawytscha | |
| Tilapia | Oreochromis niloticus | Yann et al. (1999) |
| Trout | Onchorhynchus mykiss | Yann et al. (1999) |
| amphibians: | | |
| Bull Frog | Rana catesbianca | Yu et al. (1993) |
| Reptiles: | | |
| Whiptail Lizard | C. uniparens | Wennstroms & Crews (1995) |
| Painted Turtle | Trachemys scripta | Wibbels & Crews (1994) |
| Turtle | Emys orbicularis | Dorrizi, et al. (1994) |
| Birds: | | |
| Chicken | | Shimada, et al. (1996) |
| Molluscs: | | |
| Marine snail | Nucella lapillus | Bettin, et al. (1996) |
| Marine snail | Hinia reticulata | Bettin, et al. (1996) |

### EXAMPLE 4

### Zebrafish and Medaka Rearing Protocols

Breeding and rearing protocols for zebrafish generally follow Westerfield (1995) and that of medaka follow Yamamoto (1975). Zebrafish stock was obtained from a local pet store; however, it would be appreciated by those skilled in the art that zebrafish could equally be obtained from laboratories around the world (e.g., Institute of Neuroscience, Eugene, Oreg., USA). The lf-strain of medaka was obtained from Department of Biological Sciences, University of Tokyo. Both medaka and zebrafish were maintained at 27-28 degrees C. in an in-house re-circulatory flow-through system. Embryos were obtained by natural matings, transferred into Embryo Medium (Westerfield, 1995), and incubated in a bench top incubator at 26-27 degrees C. until 3-4 days old. They were then transferred into nursery tanks maintained at 27-28 degrees C., and reared on finely ground commercial fish flakes (Tetramin), and live Artemia. After approximately 3 months, the fish were transferred into standard fish tanks alongside the adult fish. The adult fish were fed daily with flakes and occasionally supplemented with either freshly hatched or frozen Artemia.

### EXAMPLE 5

### Isolation of a Medaka Ovarian Cytochrome P450 Aromatase Promoter and Construction of GFP Expression Vector

In order to identify a good candidate promoter and/or gene for the proposed construct, the applicants examined a number of animals, both vertebrate and invertebrate. The applicants finally decided on the well-studied models for fish, the zebrafish (Brachydanio rerio) and the medaka (Oryzias latipes). These fish models were chosen as they are reasonably well characterized, and the fish are small and relatively easily bred and reared. Moreover the medaka form an ideal-background to test sex ratio manipulation studies as their sex-determining mechanism (XX/XY, male heterogametic) is well documented.

Expression of two entirely different P450 aromatase transcripts in brain and ovary of the goldfish indicate that at least two gene loci encode the P450 aomatase in fish, unlike humans (Tchoudakova and Callard 1998). This also implies that each of the P450 aromatase gene loci is regulated by separate set of tissue specific regulatory elements. Employing the native ovarian specific promoter to drive P450 aromatase blocker in the putative female offspring should ensure both spatial and temporal synchrony resulting in their masculinization/sterlization.

The medaka ovarian P450 aromatase promoter was amplified by PCR using medaka genomic DNA. Primers were designed based on the published (genebank accession #D82969) medaka ovarian P450 aromatase gene.

The primers used to amplify the medaka ovarian P450 aromatase promoter were:

| | |
|---|---|
| Forward Primer (mArom5′/1F) | |
| 5′AGTAGAGCTCAAAGACACCACCACAAAAATC--3′ | SEQ ID NO:01 |
| Reverse Primer (mArom5′/1170R) | |
| 5′-TAATGGTACCAGAAAGAAGGATGCAAGCAA-3′ | SEQ ID NO:02 |

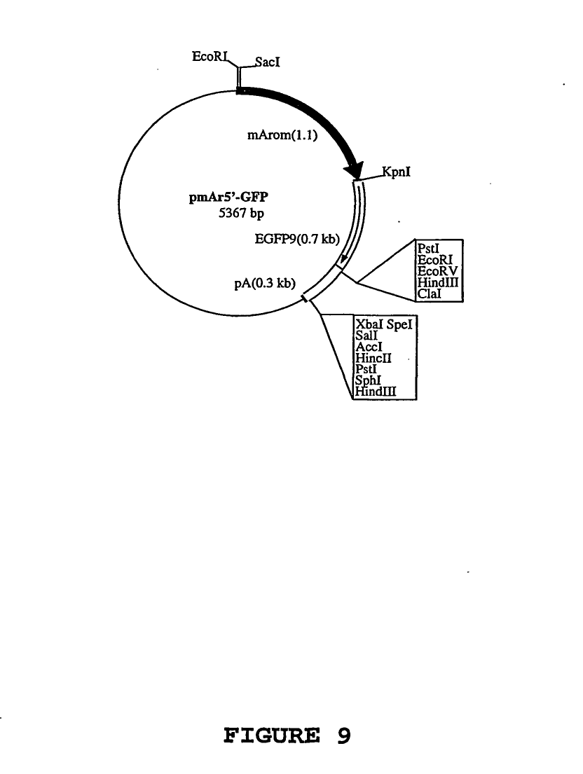

The primers were designed to carry a SacI site and a KpnI site on the 5′ and 3′ ends of the amplified product respectively. Following amplification the 1170 bp product was digested with SacI and KpnI and directionally cloned into pGEM-EGFP containing the modified GFP reporter gene (GM2, see Cormack et al., 1996) resulting in the expression-construct pmAr5′GFP (FIG. 9).

We considered that the control of ovary specific expression of P450 aromatase likely resided in this SacI-KpnI fragment, and would be useful in controlling the Sex Determining Construct. However, we are sure that any promoter-with an appropriate spatial-temporal pattern could be used in the final Sex Determining Construct.

The construct pmAr5′GFP was inserted into zebrafish embryos to test whether it conferred spatial-temporal expression pattern expected of a fish ovarian aromatase gene. This construct and all subsequent constructs were prepared using the following procedures and introduced into the developing embryos by microinjection.

All the DNA preparations were appropriately linearized and gel purified (Qiaquick Gel Extraction Kit) before injection. Needles were made from borosilicate glass capillaries with filaments (GC100TF-15, Clark Electromedical instruments) using a P-80PC micropipette puller (Sutter Instrument Co.). The needle was back-filled with purified DNA diluted to 100 ng/l in 1×injection buffer (10×; 50 mM Tris; 5 mM EDTA; 1M KCl, pH 7.2) using a hand pulled pipette. Injections were carried out on a dissection microscope fitted with two, 3-dimensional Narshige MN-151 micromanipulators. Embryos were held in place during injection by a hydraulically (mineral oil) driven holding pipette. Injection of DNA solution was facilitated pneumatically using a 3-way foot operated plunge valve (Festo Engineering), connected between the injection needle holder and nitrogen tank. Injection was performed on one-cell stage embryos, unless specifically indicated otherwise. Injected embryos were incubated and reared as described above.

Post-injection, early-stage embryos were examined under UV illumination in a Zeiss microscope equipped with standard fluorescent isothiocynate (FITC) filter set, while later-stage embryos were anaesthetized in embryo medium containing 0.125%, 2-phenoxyethanol (Sigma P-1126), before examination. Photomicrographs of embryos expressing-GFP were obtained for analysis.

Table 2 summarises the injection trials. The percentage of embryos expressing GFP at about 31 h post injection (hpi) was about 13% in both the batches tested.

TABLE 2
RESULTS OF GFP EXPRESSION IN EMBRYOS INJECTED WITH
PMAR5′ GFP AT ABOUT 30-31 H POST INJECTION

| | | | No. with | No. with |
|---|---|---|---|---|
| | | | putative | eye/anterio- |
| Batch | No. | No. with GFP | gonadal | ventral |
| No. | injected | expression | expression | expression |
| 1 | 45 | 6 | 3 | 3 |
| 2 | 23 | 3 | 1 | 2 |
| Total | 68 | 9 | 4 | 5 |

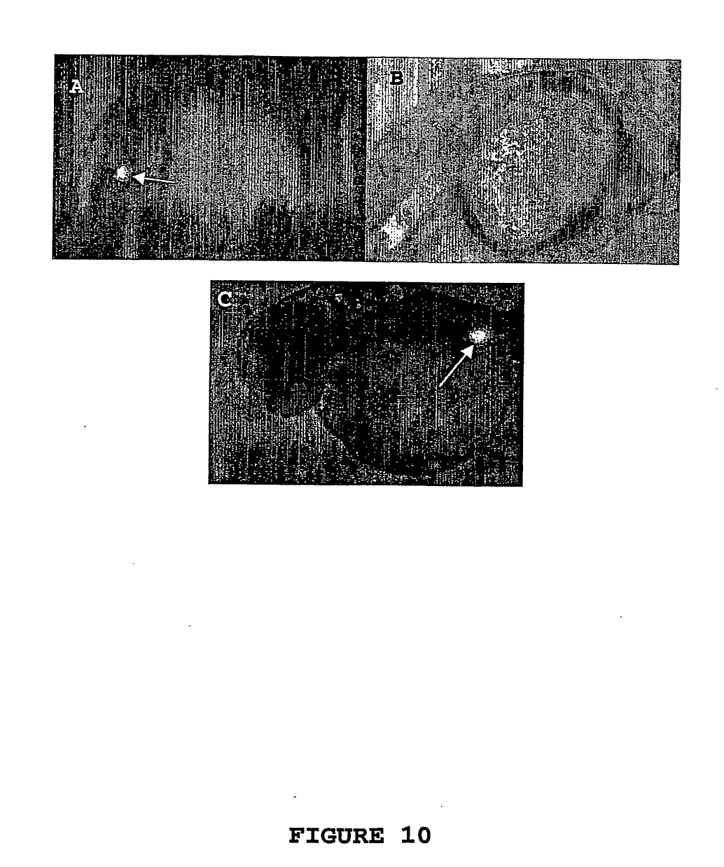

A lower than normal percentage of transient expression is expected as the promoter is expected to be active only in female embryos (see Example 9, below). First expression was detectable at about 24-25 hpi, coinciding with pharyngula. At 31 hpi, four of the nine GFP expressing embryos had expression in the ventral region located halfway along the anterior-posterior axis (FIG. 10). This region corresponds to the location of embryonic primordial germ cell clusters zebrafish during pharyngula. Of the remaining 5 individuals expressing positively, one had retinal expression and the others had expression either dorsal or ventral to the heart chamber (data not shown). Expression was persistent as late as 96 hpi in all the expressing embryos. The results indicate that the promoter fragment contains all the necessary regulatory elements required for ovarian expression of the P450 aromatase gene. None of the embryos had any brain expression, providing evidence that perhaps in both medaka and zebrafish, as in goldfish, the P450 aromatase is encoded by more than one loci.

The medaka ovarian P450 aromatase promoter sequence is shown in SEQ ID NO:03

### EXAMPLE 6

### Isolation and Cloning of Medaka and Zebrafish Ovarian P450 Aromatase cDNA

Total ovarian RNA was extracted from both zebrafish.and medaka using Trizol (Gibbco BRL) as per the instructions of the supplier. The total RNA was used as a template to generate the respective full length cDNA using the Smart cDNA kit (Clonetech). The respective total cDNA were then used as template for specific amplification of the zebrafish and medaka ovarian P450 aromatase. Primers were designed based on the published zebrafish (Genebank accession #AF004521) and medaka (Genebank accession #D82968) to encompass all of coding region.

Primers used to amplify zebrafish ovarian P450 aromatase were:

| | |
|---|---|
| Forward primer (zArom1F) | |
| 5′-ccatcgatatccgttcttatggcaggtga-3′ | SEQ ID NO:04 |
| and | |
| Reverse primer (zArom1571R) | |
| 5′-ccatcgatatgtgatggcgaatgagtgtg-3′ | SEQ ID NO:05 |

Primers used to amplify medaka ovarian P450 aromatase were:

| | |
|---|---|
| Forward primer (mAromU1) | |
| 5′-agcagctgtgctcattgttgc-3′ | SEQ ID NO:06 |
| and | |
| Reverse primer (mAromL1) | |
| 5′-atacatgaagcactggtggtc-3′ | SEQ ID NO:07 |

The specific cDNA were cloned into pGEMTeasy vector using a commercially available TA cloning kit (Promega) and their identity confirmed by sequencing. The resulting clones harbouring zebrafish and medaka ovarian P450 aromatase were designated pzArcDNA and pmArcDNA respectively (data not shown).

### EXAMPLE 7

### Combined Promoter and Blocker DNA Construct for Zebrafish P450 Ovarian Aromatase

The applicant constructed two options for blocking expression of the candidate genes in zebrafish: antisense (Izant and Weintraub 1984) and double stranded RNA (dsRNA) (Guo and Kemphues, 1995).

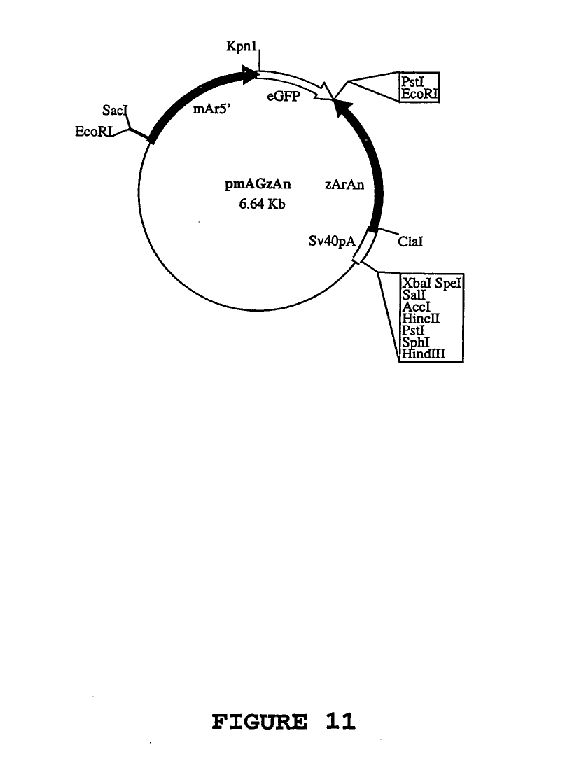

The antisense construct was made by excising a 1.291 kb zebrafish ovarian aromatase cDNA as an EcoRI and ClaI fragment from pzAr cDNA and directionally cloning it into ClaI/EcoRI linearized pmAr5′GFP. The resulting GFP-aromatase antisense fusion construct, pmA5′GzAn (FIG. 11; AGAL REF# NM00/14911), was confirmed by restriction digests and sequencing. The fusion construct was capable of co-expressing GFP and zebrafish ovarian aromatase antisense under the regulation of the medaka aromatase promoter. Co-expression of GFP with the ovarian aromatase antisense provided an easily detectable marker to distinguish the transformed embryos. The pmA5′GzAn was linearized with SacI for injection into the embryos.

The DNA sequence for the zebrafish ovarian P450 aromatase antisense is given as SEQ ID No: 8.

The double stranded aromatase blocker was constructed by ligating three molecules directionally. The first segment was a 1.8 kb fragment consisting of the medaka aromatase promoter and GFP, excised using SacI and EcoRI from pmAr5′-GFP.

The second segment was a 500 bp fragment of the zAromatase cDNA from sequence 2-502 in the published cDNA sequence (GENBANK accession AF004521; Bauer, M. P. and Goetz, F. W.). This fragment was amplified using the following primers:

| | |
|---|---|
| Forward Primer zAromX(Eco).F | |
| 5′-CCATCGAATTCCGTTCTTATGGCAGGTGA-3′ | SEQ ID NO:9 |
| Reverse Primer zAromX(Sal).R | |
| 5′-GCGTCGACTTTCTTCCAGAGAGCCACA-3′ | SEQ ID NO:10 |

The resulting amplified product had an EcoRI site on the 5′-end and a SalI site on the 3′-end for ease of cloning. The third section was a 303 bp fragment of cDNA (bases 7-309)-which was amplified using the following primers:

| | |
|---|---|
| Forward Primer zArom(Pst.)F 2 | |
| 5′-CTGCAGATATCCGTTCTTATGGCAGGTGA-3′ | SEQ ID NO:11 |
| Reverse Primer zArom(Sal).R | |
| 5′-TGCTGTTGTCGACGTTGCTGGCAGTCCC-3′ | SEQ ID NO:12 |

These primers generated a PstI site on the 5′ end and SalI site on the 3′ end for cloning. When ligated to the second fragment, the third segment formed an inverted repeat of the 5′ end of the cDNA (bases 2 through 309).

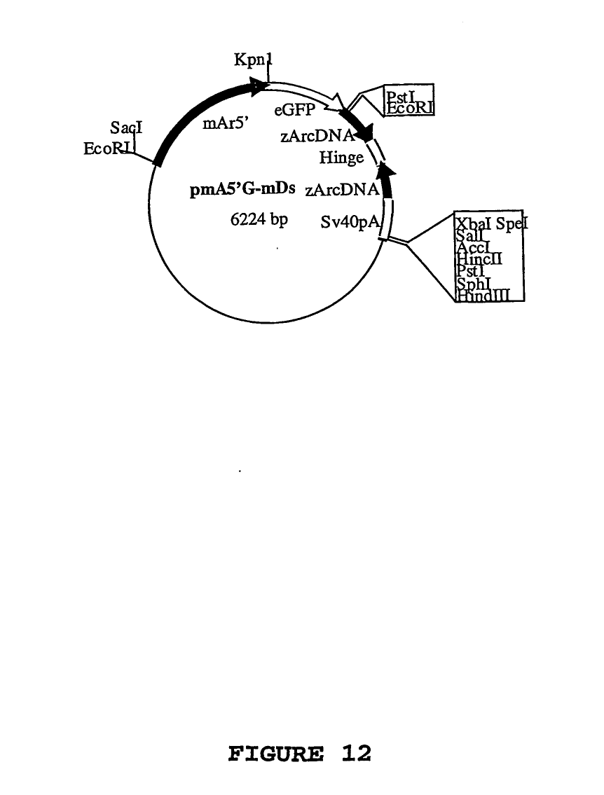

The third section, a 3 kb vector backbone, was obtained by digesting pmAr5′GFP with SacI-XbaI. The resulting fusion construct, pmA5′G-zDS (FIG. 12; AGAL REF #NM00/14907), is capable of co-expressing GFP and zebrafish ovarian aromatase double stranded DNA under the regulation of the medaka aromatase promoter. Co-expression of GFP with the ovarian aromatase blocker provided an easily detectable marker to distinguish the transformed embryos. The pmA5′G-zDs was linearized with SacI for injection into the embryos.

The DNA sequence for the double stranded promoter/blocker combination clone is given as SEQ ID NO:13. The DNA sequence for the antisense promoter/blocker combination clone is given as SEQ ID NO:14.

### EXAMPLE 9

### Experimental Test of the Effect of DS Aromatase Blocker of Phenotypic Sex in Zebrafish

In order to demonstrate the effectiveness of the genetic-construct to determine phenotypic sex, the applicants tested the construct through transient expression in zebrafish.

The construct pmAr5′GFP (FIG. 9) was inserted into zebrafish embryos to test whether it conferred spatial-temporal expression pattern expected of a fish ovarian aromatase gene and whether, as expected, it expressed only in genetic females. To test whether the aromatase blocker could produce a male phenotype, the fusion construct pmAr5′G-zDS (FIG. 12, SEQ ID. NO. 15) was also transfected into zebrafish embryos. This construct and all subsequent constructs were prepared using the following procedures and introduced into the developing embryos by microinjection.

All the DNA preparations were appropriately linearized and gel purified (Qiaquick Gel Extraction Kit) before injection. Needles were made from borosilicate glass capillaries with filaments (GC100TF-15, Clark Electromedical instruments) using a P-80PC micropipette puller (Sutter Instrument Co.). The needle was back-filled with purified DNA diluted to 100 ng/l in 1×injection buffer (10×; 50 mM Tris; 5 mM EDTA; 1M KCl, pH7.2) using a hand pulled pipette. Injections were carried out on a dissection microscope fitted with two, 3-dimensional Narshige MN-151 micromanipulators. Embryos were held in place during injection by a hydraulically (mineral oil) driven holding pipette. Injection of DNA solution was facilitated pneumatically using a 3-way foot operated plunge valve (Festo Engineering), connected between the injection needle holder and nitrogen tank. Injection was performed on one-cell stage embryos, unless specifically indicated otherwise. Injected embryos were incubated and reared as described above.

Post-injection, early-stage embryos were examined under UV illumination in a Zeiss microscope equipped with standard fluorescent isothiocynate (FITC) filter set, while later-stage embryos were anaesthetized in embryo medium containing 0.125%, 2-phenoxyethanol (Sigma P-1126), before examination. Photomicrographs of embryos expressing GFP were obtained for analysis.

Sex was determined by microscopic examination, following criteria described by Satoh and Egami (1972), among others. Standard histological procedures, generally following Humason (1979), were used to prepare and examine juveniles. In brief, the juveniles were netted out of their holding tanks and euthanased in a 1 ppm 2-phenoxyethanol solution. The fish were then immediately fixed whole in 10% neutral buffered formalin. The tissue was processed routinely for paraffin wax infiltration and saggital sections (4 μm) were stained with haematoxylin and eosin. Histological sections were examined under standard illumination using a Leitz microscope (100-400×). Ovaries were identified as elongate organs located between the gut and swim bladder, adjacent to the liver, and characterised by a pavement-like structure with large, heavily stained cells and, in larger juveniles, the beginnings of an ovarian lumen. Because of their early ontogenetic development, ovaries are usually easy to identify in all but very slow growing 30-day post-hatch zebrafish. Testes have a fine, granular structure, develop more slowly than ovaries, and are difficult to discern in 30-day post-hatch juveniles.

Table 3 summarises two injection trials using pmAr5′GFP. The percentage of embryos expressing GFP at about 31 h post injection (hpi), were about 30-40%.

TABLE 3
PHENOTYPIC SEX OF 30 D POST-HATCH JUVENILES
EXPRESSING pMAR5′GFP AT ABOUT 30-31 H POST INJECTION

| | | No. with | No. with | | |
|---|---|---|---|---|---|
| | | GFP | putative gonadal | | |
| | | expression | expression | Number of | Number of |
| Batch | Number | in putative | examined for | Examined | Examined |
| No. | injected | gonad | phenotypic sex | Females | Males |
| 1 | 73 | 27 | 5 | 5 | 0 |
| 2 | 86 | 23 | 6 | 6 | 0 |
| Total | 159 | 60 | 11 | 11 | 0 |

At 30 days post-hatching eleven juveniles that expressed GFP in their putative gonads were selected randomly for determination of their sex. All eleven had conspicuous ovaries. The probability by chance alone that all 11 juveniles examined would be female, given an assumed initial sex ratio in injected embryos of 50:50 male:female, is 1 over 2¹¹, which is less than 0.001%. We conclude from this experiment that pmArom expresses only in genetic females in zebrafish.

On the basis of this experiment, we conclude that even one male individual in a blocking construct driven by pmArom provides evidence that the invention functions as predicted.

In subsequent trials, sex was determined either as described above, or by macroscopic inspection of the individuals. Specifically, fish to be sexed were sedated to anaesthetic stage two in a solution of 2-phenoxyethanol. They were then removed from the anaesthetic bath solution and placed into a slot cut into a block of wetted polyurethane foam. The slot held the fish still with its ventral surface exposed. The vent was blotted dry with a paper towel to ensure any expressed sperm is not activated. The exposed abdomen was gently squeezed with a pair of fine forceps, tipped with plastic boots, to express gametes. The gametes were collected in glass capillary tube from the vent. The contents of the capillary tube were emptied into a drop of water on a microscope slide and examined under a compound microscope at 400×magnification. Sperm was conspicuous when present.

Table 4 summarises an injection trial using pmAr5′G-zDS fusion construct. Low fertilisation rates resulted in low embryonic survival and hence very small numbers of juveniles for examination.

TABLE 4
PHENOTYPIC SEX OF 30 D POST-HATCH JUVENILES
EXPRESSING GFP FROM THE pMAR5′G-ZDS FUSION
CONSTRUCT AT ABOUT 30-31 H POST INJECTION

| | | No. with | No. with | | |
|---|---|---|---|---|---|
| | | GFP | putative gonadal | | |
| | | expression | expression | Number of | Number of |
| Batch | Number | in putative | examined for | Examined | Examined |
| No. | injected | gonad | phenotypic sex | Females | Males |
| 1 | 359 | 23 | 13 | 3 | 10 |
| 2 | 385 | 20 | 5 | 0 | 5 |

In the first experiment, three animals were sacrificed at 4 weeks age post-hatch to assess their phenotypic sex. Two were clearly female, with well developed, conspicuous ovaries. The third lacked ovaries. A small, elongate and densely stained structure, located between the gut and swim bladder, was identified as an early stage testes. The male sexuality of the specimen was independently confirmed by a specialist fish pathologist from the University of Tasmania.

The remaining fish in batch 1 and all fish in batch 2 were reared to an age of 3 months, and then assayed for sex by gently squeezing the abdomen to cause extrusion of eggs or sperm, as described above. The viability of the extruded material was assayed by examination under a dissecting microscope. In batch 1, of the ten remaining animals that had expressed GFP in the gonad, 9 were males (with actively swimming sperm) and one was female. In batch two, all five animals that had expressed GFP produced actively swimming sperm.

The large majority of fish expressing the pmAr5′G-zDS fusion construct verifies the predicted action of the invention on sex differentiation. Moreover, over the two experiments, the difference between the sex ratio of fish expressing pmAr5′G-zDS fusion construct was significantly different from fish expressing just the pzfAr5′GFP (18 males: 3 females, versus 0 males:11 females) (Chi Square=22.6, df=1, p<0.001) and also different from an assumed 50:50 sex ratio (Chi square=4.01, df=1, p<0.05).

Three males from batch 1 were subsequently paired with wild-type females. All courted as typical for zebrafish, and all subsequently spawned with the wild-type females and produced fertile eggs.

We conclude from these experiments 1) that the aromatase-promoter only expresses in genetic females, 2) that the pmAr5″G-zDS construct resulted in genetic females developing into a male phenotype, and 3) that the sex-changed individuals were reproductively viable as males. The inconsistent action of the construct is not surprising at this stage, given likely differences among specimens in copy number and a probable dose-dependent response.

### EXAMPLE 10

### A Repressible Aromatase Blocker

For breeding purposes, it may be useful to temporarily repress the aromatase blocker, as to produce normal females carrying the sex differentiating construct. To achieve this, the applicants used a commercially available repressible element as the externally keyed genetic switch or Tet-responsive P_{hCMV*-1} promoter. P_{hCMV*-1} contains the Tet-responsive element (TRE) which consists of seven copies of the 42 bp tet operator sequence (tetO). This element is just upstream of the minimal CMV promoter (P_{minCMV}), which lacks the enhancer that is part of the complete CMV promoter. Therefore, P_{hCMV*-1} is silent in the absence of binding of transactivator protein (tTA) to the tetO. The tetracycline-sensitive element is described by Gossen and Bujard (1992; tet-off), Gossen et al. (1995; Tet-on), and Kistner et al. (1996). In the tetracycline-regulated system (Tet-Off system) developed by Hermann Bujard, addition of tetracycline (Tc) or doxycycline Dox; a Tc derivative) prevents the binding of a tTA, to the Tet-responsive element. This then blocks gene expression from the TRE until the drug is removed. A complementary system has also been developed (Tet-On system). In the Tet-On system, addition of doxycycline allows the binding of a reverse transactivater, rtTA, to the tetO promoter, leading to gene expression from the TRE. Gene expression continues from the TRE until removal of the drug. A tetracycline responsive element has the advantage of ease of administering. Tetracycline is a routinely used antibiotic in fish and shellfish culture (see Stoffregan et al., 1996), readily traverses cutaneous membranes while retaining its biological activity, and can be administered by whole organism immersion. Use of the Tet-On/Off controllable expression systems is covered by U.S. Pat. No. 5,464,758, assigned to BASF Aktiengesellschaft.

The Tet-On™ and Tet-off™ gene expression system and the Tet responsive bidirectional vectors pBI and pBI-GFP were purchased from a commercial source (Clontech). The pzBMP2-Tet-Off construct was engineered by excising PminCMV promoter as SpeI and EcoRI fragment from pTet-Off and replacing it with the 1,414 bp zBMP2 promoter as XbaI/EcoRI, from pzBMP2-(1.4), by directional cloning Sequence AGAL REF# NM99/09099).

A single construct was developed which incorporates expression of the tTA under control of the spatially and temporally defined aromatase promoter. Expressed tTA complex then binds to the TRE (tet responsive element) resulting in expression of GFP and a second gene of interest which has been cloned into the multiple cloning site (MCS). This second gene ideally encodes a blocker sequence such as antisense or double stranded RNA. Thus, regulation of the blocker sequence and eGFP are under control of the spatially and temporally regulated promoter via activation of the TRE.

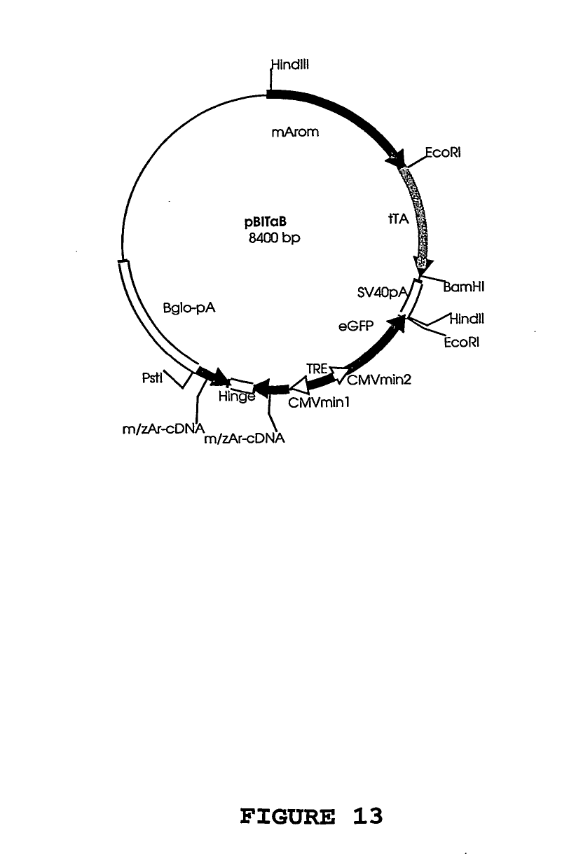

Three elements were brought together to form this construct (FIG. 13). The first element utilized the pGI-eGFP vector supplied by Clontech which was modified to achieve the plasmid pBi(-SV40) following excision of the SV40 Poly A segment using ApaI and SalI. Resulting ends were then filled in with T4 DNA polymerase followed by religation to reconstitute a viable plasmid.

The second element was generated by excising the medaka aromatase promoter and tTA coding sequence from the pmAr5′-Tet-Off plasmid using HindIII. This fragment was then inserted into the HindIII site of pBi(-SV40) resulting in a second construct pBi-mArtTA.

The third element consisted of an EcoRI-HindIII fragment-containing either a zebrafish dsRNA aromatase blocker or one from medaka. Ends of the EcoRI-HindIII fragment were filled in with T4DNA polymerase and this fragment was ligated into the PvuII site of pBi-BMPtTA This resulted in a final construct capable of repressibly blocking zebrafish aromatase (pBiT-ds.zAroma; SEQ ID NO.16).

### REFERENCES

Ausubel, F. M., Brent, R., Kingston, R. E., Moore, D. D., Sridman, J. G., Smith J. A. and Struhl, K. (Eds.; 1996). Current protocols in molecular biology. John Wiley and Sons Inc, USA. Vol 1-3.

Bettin, C., Oehlmann, J. and Stroben, E. (1996). TBT-induced imposex in marine neogastropods is mediated by an increasing androgen level. Helgolander Merresuntersuchungen 50:299-317.

Cormack B P, Valdivia R H, Falkow S (1996)FACS-optimised mutants of the green fluorescent protein (GFP). Gene 173:33-38.

Dale, L., Howes, G., Price, B. M. J. and Smith J. C. (1992) Bone morphogenetic protein 4: a ventralizing factor in early Xenopus development. Development 115:573-585

DeRobertis, E. M. and Sasai, Y (1996). A common plan for dorsoventral patterning in bilateria. Nature 380:37-40.

Dorizzi, M., Mignot, T. M., Guichard, A., Desvages, G., and Pieau, C. (1991). Involvement of estrogens in sexual differentiation of gonads as a function of temperature in turtles. Differentiation 47:9-17.

Driever et al (1996). A genetic screen for mutations affecting embryogenesis in zebrafish. Development. 123:37-46

Elbrecht, A. and Smith, R. G. (1992). Aromatase enzyme activity and sex determination in chickens. Science 255:467-470.

Ferguson, E. L. and Anderson, K. V. (1992). Decapentaplegic acts as a morphogen to organize dorsal-ventral pattern in the Drosophila embryo.Cell 71:451-461.

Fire, A., Xu, S., Montogomery M. K., Kostas, S. A. Driver, S. E. and Mello C. C. (1998). Potent and specific genetic interference by double-stranded RNA in Caenorhabditis elegans. Nature.391:806-811.

Francois, V., Solloway, M., O'Neill, J. W., Emery, J. and Bier, E. (1994) Dorsal-ventral patterning of the Drosophila embryo depends on a putative negative growth factor encoded by the short gastrulation gene. Genes Dev 8:2602-2616

Gibbs, P. E., Pascoe, P. L. and Burt, G. R. 1988. Sex change in the female dog-whelk, Nucella lapillus, induced by tributyltin from antifouling paints. J. Mar. Biol. Assoc. UK 68:715-731.

Gossen, M. & Bujard, H. (1992) Tight control of gene expression in mammalian cells by tetracycline responsive promoters. Proc. Natl. Acad. Sci. USA 89:5547-5551.

Gossen, M., Freundlieb, S., Bender, G., Muller, G., Hillen, W. & Bujard, H. (1995) Transcriptional activation by tetracycline in mammalian cells. Science 268:1766-1769.

Guiguen, Y., Baroiller, J-F., Ricordel, M-J., Iseki, K., McMeel, O., Martin, S. And Fostier, A. (1999). Involvement of estrogens in the process of sex differentiation in two fish species: the rainbow trout (Oncorhynchus mykiss) and a tilapia (Oreochromis niloticus). Molcular Reprod. Development 54:154-162.

Hafter et al (1996). The identification of genes with unique and essential functions in the development of the zebrafish, Danio rerio. Development. 123:1-36.

Hamilton, W. D. (1967). Extraordinary sex ratios. Science 156:477-489.

Hammerschmidt, M., Serbedzija, N. and McMahon, A. P. (1996) genetic analysis of dorsoventral pattern formation in the zebrafish: requirement of a BMP-like ventralizing activity and its dorsal repressor. Genes Dev. 10:2452-2461.

Hines, G. A., Watts, S. A., Walker, C. W. and Voogt, P. A. (1992) Androgen metabolism in somatic and germinal tissues of the sea star Asterias vulgaris. Comp. Biochem. Physiol. 102B:521-526.

Holley, S. A., Jackson, P. D., Sasai, Y., Lu, B., De Robertis, E. M., Hoffmann, F. M., and Ferguson, E. L. (1995) A conserved system for dorsal-ventral patterning in insects and Animals involving sog and chordin. Nature 376: 249-253.

Humason, G. L. (1979). Animal Tissue Techniques (Fouth Edition). Freeman, San Francisco. 661 Pp.

Izant J G, Weintraub H (1984). Inhibition of thymidine kinase gene expression by anti-sense RNA: a molecular approach to genetic analysis. Cell 36:1007-1015.

Jones, C. M., Dale, L., Hogan, B. L. M., Wright C. V. E. and Smith, J. C. (1996) Bone morphogenetic protein-4 (BMP4) acts during gastrula stages to cause ventralization of Xenopus embryos. Development 122:1545-1554.

Kishimoto, Y., Lee, K. H., Zon, L., Hammerschmidt, M. and Schulte-Merker, S. (1997). The molecular nature of zebrafish swirl: BMP2 function is essential during early dorsoventral patterning. Development. 124:4457-4466.

Kistner A, Gossen M, Zimmermann F, Jerecic J, Ullmer C, Lubbert H, and Bujard H. (1996) Doxycycline-mediated quantitative and tissue-specific control of gene expression in transgenic mice. Proc Natl Acad Sci USA 93:10933-10938.

Lardans, V. and Dissous C. (1998). Snail control strategies for reduction of schistosomiasis transmission. Parsitology Today 14:413-417.

Lemaire,. P. and Yasuo H (1998). Developmental signalling: a careful balancing act. Curr Biol.8:228-231

Li, Y-X, Farrell, M. J., Liu, R., Mohanty, N. And Kirby, M. L. (2000). Double-stranded RNA injection produces null phenotypes in Zebrafish. Develop. Biol., 217: 394-405.

Matthiessen, P and Gibbs, P. E. (1998). Critical appraisal of the evidence for tributyltin-mediated disruption in mollusks. Environ. Toxicol. Chem. 17:37-43.

Matsumoto, T., Osada, M., Osawa, Y. And Mori, K. (1997). Gonadal estrogen profile and immunohistochemical localisation of steroiogenic enzymes in the oyster and scallop during sexual maturation. Comp. Biochem. Physiol.,B, 118B:811-817.

Mishihina, Y., Susuki, A., Ueno. N. and Behringer, R. R. (1995). BMPr encodes a type I bone morphogenetic protein receptor that is essential for gastrulation during mouse embryogenesis. Genes and Dev. 9:3027-3037.

Miya, T., Morita, K., Suzuki, A., Ueno, N. and Satoh, N. (1997). Functional analysis of an ascidian homologue of Animal Bmp-2/Bmp-4 suggests its role in the inhibition of neural fate specification.Development.124:5149-5159

Oderdorster, E. (1997). Sublethal effects of tributyltin on aquatic invertebrate models:steroid metabolism, protein alterations and physiological responses. Thesis, Duke University, 1156 Pp.

Piccolo, S., Sasai, Y., Lu, B. and DeRobertis, E. M. (1996). A possible molecular mechanism for Spemann organizer function: Inhibition of ventral signals by direct binding of Chordin to BMP-4. Cell 85:589-598.

Piferrer, F., Zanuy, S., Carrillo, M., Solar, I., Devlin, R. H. and Donaldson, E. (1994). Breif treatment with an aromatase inhibitor during sex differentiation causes chromosaomally female salmon to develop as normal, functional males. J. Exper. Zool. 270:255-262.

Sambrook, J., Fritsch, E. F. and Maniatis, T. (eds.)(1989). Molecular Cloning A Laboratory Manual, 2nd Ed., Cold Spring Harbor Laboratory Press. Vol 1-3.

Sator, N. and Egami, N. (1972). Sex differentiation of germ cells in the teleost, Oryzius latipes, during normal embryonic development. J. Embryol. Exp. Morph. 28:385-395.

Schmidt, J., Francois, V., Bier, E. and Kimelman, D. (1995). Drosophila short gastrulation induces an ectopic axis in Xenopus: evidence for conserved mechanisms of dorsal-ventral patterning. Development 121:4319-4328

Schulte-Merker, S., Lee, K., McMahon A. P. and Hammerschmidt, M. (1997). The zebrafish organizer requires chordino. Nature 387:862-863.

Stoffregen, D. A., Bowser, P. R. and Babish J. G. (1996). Antibacterial chaemotherapeutants for finfish aquaculture: A synopsis of laboratory and field efficacy and saftey studies. J. Aquatic Animal Health. 8:181-207.

Suzuki Y, Yandell M D, Roy P J, Krishna S, Savage-Dunn C, Ross R M, Padgett R W, and Wood W B (1999). A BMP homolog acts as a dose-dependent regulator of body size and male tail patterning in Caenorhabditis elegans. Development 126(2): 241-250

Tjian R. and Maniatis T (1994). Transcriptional activation: a complex puzzle with few easy pieces. Cell 8:77:5-8.

Waterhouse, P., Graham, M. W. and Wang, M-B. (1998). Virus resistance and gene silencing in plants can be induced by simultaneous expression of sense and antisense RNA. Proc. Natl. Acad. Sci. 95:13959-13964.

Wennstrom, K. L. and Crews, D. (1995). Making males from females: the effects of aromatase inhibitors on a parthenogenic species of whiptail lizard. Gen. Comp. Endocrinol. 99:316-322.

Werren, J. H., Skinner, S. W. and Charnov, E. L. (1983). Paternal inheritance of a daughterless sex ratio factor. Nature 293:467-468.

Westerfield M. (1995). The zebrafish book: University of Oregon Press.

Wibbels, T. and Crews, D. (1994). Putative aromatase inhibitor inuces male sex determination in a female unisexual lizard and a turtle with temperature-dependent sex determination. J. Endocrinol. 141:295-299.

Yu, N-W., Hsu, C-Y, Ku, H-H, Chang, L-T and Lu, H-W. (1993). Gonadal differentiation and secretions of estradiol and testosterone of the ovaries of Rana catebianca tadpoles treated with 4-hydroxyandrostenedione. J. Exper. Zool. 265:252-257.

The claims defining the invention are as follows:

1. A construct for modifying phenotypic sex in animals, comprising:
a) a first nucleic acid molecule, which is transiently activated in a defined spatio-temporal pattern, and which is operably linked to
b) a second nucleic acid molecule, which encodes a blocker molecule that alters normal sexual development in the animal.

2. A construct according to claim 1, wherein each of the first and second nucleic acid molecules is either genomic DNA, cDNA, RNA, or a hybrid molecule thereof.

3. A construct according to claim 1, wherein each of the first and second nucleic acid molecules are full-length molecules, or biologically active fragments thereof.

4. A construct according to claim 1, wherein the first nucleic acid molecule is a DNA molecule encoding a promoter region.

5. A construct according to claim 4, wherein the promoter is activated only during embryonic development and/or gametogenesis, and is expressed in a spatio-temporal domain coincident with sex determination.

6. A construct according to claim 5, wherein the promoter has the nucleotide sequence shown in SEQ ID NO:3.

7. A construct according to claim 1, wherein the second nucleic acid molecule encodes a blocker molecule selected from the group consisting of antisense RNA, double-stranded RNA (dsRNA), sense RNA and ribozyme.

8. A construct according to claim 7, wherein the second nucleic acid molecule is antisense RNA or dsRNA.

9. A construct according to claim 7, wherein the second nucleic acid molecule has the nucleotide sequence shown in SEQ ID NO:8 or SEQ ID NO:13.

10. A construct according to claim 1, as deposited under the Budapest Treaty at the Australian Government Analytical Laboratories and accorded the accession numbers NM00/14911 or NM00/14907.

11. A nucleic acid molecule which encodes the coding region of a gene including:
a) a nucleotide sequence as shown in SEQ ID NO:13;
b) a biologically active fragment of the sequence in a); or
c) a nucleic acid molecule which has at least 75% sequence homology with the sequence disclosed in a) or b); or
d) a nucleic acid molecule that is capable of binding to the sequences disclosed in a) or b) under stringent conditions.

12. A nucleic acid molecule which encodes a blocker molecule, wherein the blocker molecule is capable of altering normal sexual development in an animal, leading to sterility or an alteration of phenotypic sex.

13. A nucleic acid molecule according to claim 12, wherein the blocker molecule is selected from the group consisting of antisense RNA, dsRNA, sense RNA and ribozyme.

14. A nucleic acid molecule according to claim 12, wherein the blocker molecule is dsRNA.

15. A nucleic acid molecule according to claim 12, wherein the blocker molecule is encoded, or partially encoded, by a nucleic acid sequence shown in SEQ ID NO:8 or SEQ ID NO:13.

16. A method of altering phenotypic sex in animals comprising the steps of:
1) stably transforming an animal cell with a construct according to any one of claims 1 to 10; and
2) implanting the cell into a host organism, whereby a whole animal develops from the implanted cell.

17. A method according to claim 16, wherein the stable transformation is effected by microinjection, transfection or infection, wherein the construct stably integrates into the genome by homologous recombination.

18. A method according to claim 16, wherein the host organism is of the same genus as the transformed cell.

19. A method according to claim 16, wherein the host organism is a vertebrate.

20. A method according to claim 16, wherein the host organism is an invertebrate.

21. A method according to claim 16, wherein the host organism is selected from the group consisting of fish, amphibians and molluscs.

22. A method according to claim 21, wherein the fish is selected from the group consisting of zebrafish, European carp, salmon, mosquito fish, tench, lampreys, round gobies, tilapia and trout.

23. A method according to claim 21, wherein the amphibian is selected from the group consisting of cane toads and bull frogs.

24. A method according to claim 21, wherein the mollusc is selected from the group consisting of Pacific oysters, zebra mussels, striped mussels, New Zealand screw shells, the Golden Apple Snail and Giant African Snail, or a snail from the genera Biomphalaria or Bulinus.

25. A transgenic animal made by a method according to claim 16.
